(19) United States
(12) Patent Application Publication
(10) Pub. No.: US 2026/0192296 A1
(43) Pub. Date: Jul. 9, 2026

(54) DEVICES, SYSTEMS, AND METHODS FOR PROCESSING BIOLOGICAL SAMPLES USING ISOTACHOPHORESIS

(71) Applicant: LIFE TECHNOLOGIES CORPORATION, Carlsbad, CA (US)

(72) Inventors: I-Jane Chen, Pleasanton, CA (US); Nestor Castillo, San Leandro, CA (US); Robert Green, Cupertino, CA (US); Liansen Liu, Redwood City, CA (US)

(21) Appl. No.: 19/121,952

(22) PCT Filed: Oct. 16, 2023
(86) PCT No.: PCT/US2023/076977

§ 371 Date: Apr. 17, 2025

Related U.S. Application Data
(60) Provisional application No. 63/417,867, filed on Oct. 20, 2022.

(51) Int. Cl. B01L 3/00; C12Q 1/6806

(52) U.S. Cl. CPC B01L 3/502753; B01L 3/502715; C12Q 1/6806; B01L 2200/0647; B01L 2300/0645; B01L 2300/0867; B01L 2400/0421

(57) ABSTRACT

A fluidic device for performing isotachophoresis comprise a sub-strate defining a fluidic network comprising a plurality of inlet channels, the outlets of the inlet channels meeting at a juncture, an intermediate channel extending from the juncture and fluidically coupled to the outlets of the plurality of inlet channels, a first reservoir at the juncture and fluidically coupled to the inlet channels and the intermediate channel, a second reservoir fluidically coupled to the intermediate channel at a location downstream of the juncture, wherein the inlet channels, the collection channel, and the intermediate channel are co-planar, and wherein the first reservoir and second reservoir are configured to produce substantially equal pressure heads. Methods of loading the fluidic device for isotachophoresis can be sequentially through the various inlet channels.

### CROSS-REFERENCE TO RELATED APPLICATIONS

This application claims the benefit of U.S. Provisional application Ser. No. 63/417,867 filed on Oct. 20, 2022. To the extent permitted in applicable jurisdictions, the entire contents of this application are incorporated herein by reference.

### TECHNICAL FIELD

Aspects of the present disclosure relate to devices, systems, and methods for processing biological samples containing ionic analytes. More specifically, aspects of the present disclosure relate to devices, systems, and methods implementing isotachophoresis to process biological samples containing ionic analytes.

### BACKGROUND

A variety of techniques exist for the analysis of components, such as but not limited to deoxyribonucleic acid (DNA), ribonucleic acid (RNA), and proteins, found in various biological samples. Analysis of such components occurs in a variety of applications, including but not limited to life science research, clinical diagnostics, and forensic analysis. These components of interests can be extracted from a raw sample, such as cells or other bodily fluids (for example, blood, urine, feces, sputum, sperm, etc.) which contain other components that may interfere with processing and/or analysis of the components of interest once extracted. For example, various assays, such as but not limited to nucleic acid amplification assays (e.g., polymerase chain reaction (PCR)), nucleic acid sequencing assays, and enzyme-linked immunoassays (ELISA)), may be subject to interference in the presence of components found in cells and other bodily fluids. Accordingly, prior to performing such assays, purification of the sample may be performed to separate the component(s) of interest from the other interfering components in the sample. Solid phase extraction (SPE), gel electrophoresis, chromatography, and isotachophoresis (ITP) are various sample purification techniques that can be used.

Another issue that arises in analysis of biological components of interest from a raw sample is an insufficient concentration of components of interests that hinders and/or prevents analysis assays to be performed. To address this issue, enrichment (increasing the concentration) of components of interest may be performed. SPE and ITP are nonlimiting techniques for enrichment.

In some cases, ITP can provide more favorable conditions than SPE for purification and enrichment, particularly when processing raw samples containing small amounts of an analyte of interest. For example, SPE techniques typically rely on a several-step process (e.g., conditioning (solvation) of the adsorbent, sample application (adsorption) washing, and elution) and achieves around a 60% yield of the analyte of interest. ITP relies on the ionic mobility differences between positively or negatively charged compounds of interests (ionic analytes) and the compounds not of interests (impurities), which can be charged or neutral, and in contrast to SPE follows a procedure with fewer steps and that can achieve close to 100% yield of the analyte of interest. When placing the sample containing the ionic analytes and impurities in an electric field, the various ionic components respond to the electric field and migrate according to polarities, e.g. a negatively charged molecule migrates toward an anode and away from a cathode, and vice versa for a positively charged molecule. ITP also relies on using a leading electrolyte (LE) comprising ions of higher ionic mobility than the ionic analyte placed between the ionic analyte and one electrode (e.g. leading electrolyte of higher ionic mobility is placed between sample containing negatively charged ionic analyte and an anode) and a trailing electrolyte (TE) comprising ions of lower ionic mobility than the ionic analyte placed between the ionic analyte and the electrode of opposite polarity (e.g. trailing electrolyte of lower ionic mobility is placed between sample containing negatively charge ionic analyte and cathode). Further, the distinct conductivity differences between leading electrolyte and ionic components of the sample and between the ionic components of the sample and trailing electrolyte form relatively steep electric field gradients at the respective interfaces between the leading electrolyte and sample and between the sample and the trailing electrolyte. These electric field gradients can further act to concentrate the various ionic components in the sample as they migrate via the electric field, thereby resulting in the rearrangement of the distribution of the various ionic components depending on the ionic mobility of each.

As depicted schematically in FIG. 1, ITP can be achieved using small volumes of sample and electrolytes in small-scale fluidic devices on the micro-or lower scale by utilizing a separation channel 1000 to contain the sample S in contact with the leading and trailing electrolytes LE, TE with an electric field (represented by the respective +/−symbols) using electrodes to create an electric field applied across the channel so as to cause migration and separation of the differing ionic components in the channel 1000. Those having ordinary skill in the art would appreciate that various configurations can be used for a separation channel and the locations of the trailing electrolyte, leading electrolyte, and sample relative to the separation channel and each other, and the applied electric field depending on the charged components of interest and FIG. 1 is intended to provide a general understanding of ITP at a conceptual level.

While ITP presents a robust technique for performing enrichment and purification and provides a relatively large yield of analyte, implementing ITP on relatively small raw sample volumes, such as via small-scale fluidic devices on the micro-or lower scale which comprise a separation channel along which the electric field operates, can present various challenges. One set of challenges relates to the effects on fluid behavior in the fluidic device during the application of the relatively high voltages to generate the electric field. For example, undesirable gaseous pressure within the fluidic device can result from the generation of gases at electrode surfaces due to electrochemical oxidation and reduction of reagents during the application of the relatively high voltages to generate the electric field. Such gaseous pressure can induce undesired motion of bulk fluid if not vented. Venting locations of the device to atmosphere, however, can make the fluidic device susceptible to hydrostatic pressure-induced bulk fluid motion, such as bulk fluid to flow in a separation channel. Another issue that can arise during the application of the relatively high voltage is Joule heating, which can elevate the temperature of fluids in the separation channel. As such, any air bubbles that may be trapped in the fluids or at fluid interfaces can be thermally expanded, which in turn can result in undesired bulk fluidic motion, current disruptions (drops), a skewed analyte distribution shape, and/or undesirable mixing of reagents, among other things. Uneven work surfaces and/or surface profiles on the fluidic device itself can further compound these issues due to the small-scale flow channels and relatively long length of the separation channel.

Other issues that can arise in ITP using small-scale fluidic devices relate to the overall separation and enrichment that are achieved so as to be able to collect the ionic analyte of interest. One issue along these lines relates to hyperbolic flow rate profiles that occur due to hydrodynamic injection as sample and/or reagents are loaded into the separation channel. As can be seen from the schematic, conceptual illustration in FIGS. 2A-2C, as an upstream fluid UF moves into the separation channel 1000 under such a hyperbolic flow rate profile, the interface of the upstream fluid UF with the downstream fluid DF containing ionic components (depicted by P) deform from a generally flat (e.g., rectangular) shaped distribution as depicted in FIG. 2A to a curved (crescent-like) shape as depicted in FIG. 2B. In other words, the interface between two fluids DF/UF deforms from a flat planar interface to a curved planar interface. Under the profile of FIG. 2B and during application of the electric field, a radial concentration gradient exists that will cause ionic components P of the downstream fluid DF to diffuse radially (depicted by arrows R) along the gradient, ultimately resulting in loss of a uniform and discrete banding of the ionic particles P as depicted in FIG. 2C. For example, the leading electrolyte may be subject to diffusion into the sample. Such impact causes a drop of the electric field difference between the upstream and downstream fluids (e.g., LE and sample), which can therefore compromise the enrichment efficiency and the ability to obtain a sufficient banding of the ionic analyte of interest.

Finally, with samples having sufficiently low concentrations of an ionic analyte, the enrichment achieved using ITP, particularly when implemented using small-scale microfluidic devices, can pose challenges for detection of the ionic analyte. In some implementations, collection of the ionic analyte is triggered via a detection technique (e.g., optical or electrical detection) that may not be triggered when concentration of the ionic analyte, even after being purified and enriched in the separation channel, is sufficiently low.

There exists a need, therefore, to provide devices, systems, and methods for performing ITP that are able to achieve robust purification and enrichment of samples containing ionic analytes that can alleviate hydrostatic pressure issues, achieve relatively good separation and enrichment efficiency, and/or address concerns of detection when low concentration of ionic analyte exists after enrichment.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are for example and explanatory only and are not restrictive of the claims; rather the claims should be entitled to their full breadth of scope, including equivalents.

### BRIEF DESCRIPTION OF DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation.

FIG. 1 is a schematic illustration depicting a channel and implementation of isotachophoresis at a conceptual level.

FIGS. 2A-2C are schematic illustrations depicting effects on isotachophoresis for a hyperbolic fluid flow of an upstream fluid on a downstream fluid.

FIGS. 3A and 3B are schematic plan and side views of an embodiment of a fluidic device for performing sample purification and enrichment using ITP.

FIG. 4A is a perspective view of a first portion of the fluidic device of FIGS. 3A and 3B comprising electrolyte reservoirs and through holes.

FIG. 4B is a perspective view of a second portion of the fluidic device of FIGS. 3A and 3B comprising a channel.

FIGS. 5A-5D schematically depict an embodiment of a workflow to use the fluidic device of FIGS. 3A and 3B to perform sample purification and enrichment using ITP.

FIGS. 6A and 6B are schematic plan and side views of another embodiment of a fluidic device for performing sample purification and enrichment using ITP.

FIG. 7A is a perspective view of a first portion of the fluidic device of FIGS. 6A and 6B comprising ports, electrolyte reservoirs, and through holes.

FIG. 7B is a perspective view of a second portion of the fluidic device of FIGS. 6A and 6B comprising channels.

FIGS. 8A-8G schematically depict an embodiment of a workflow to use the fluidic device of FIGS. 6A and 6B to perform sample purification and enrichment using ITP.

FIG. 9 is a schematic plan view of another embodiment of a fluidic device for performing sample purification and enrichment using ITP.

FIG. 10A is a perspective view of a first portion of the fluidic device of FIG. 9 comprising ports, reservoirs, and through holes.

FIG. 10B is a perspective view of a second portion of the fluidic device of FIG. 9 comprising channels.

FIGS. 11A-11G schematically depict an embodiment of a workflow to use the fluidic device of FIG. 9 to perform sample purification and enrichment using ITP.

FIG. 12 is a block diagram illustrating and embodiment of a system integrating a fluidic device for performing sample purification and enrichment using ITP in accordance with embodiments of the present disclosure.

FIG. 13 is a graph of concentration versus length in a separation channel for different ionic components (i.e., leading electrolyte, trailing electrolyte, sample containing mixture of ionic components) from a simulation of ITP.

FIGS. 14A-14F is an image of a detection region of a fluidic chip from a test run performing ITP in the fluidic chip.

FIG. 15 is an image of a detection region of a fluidic chip from a test run performing ITP in the fluidic chip.

FIG. 16 is a graph of detected fluorescence versus time from the test run of FIG. 15.

FIG. 17 is a graph of detected fluorescence versus concentration loading known concentrations of DNA.

FIGS. 18A-18C are images of a detection region from tests run performing ITP in a prototype fluidic device on differing initial blood sample concentrations and loading amounts.

FIGS. 19A and 19B are electropherograms from tests of a control sample subject to a PCR amplification assay and of a purified and enriched sample collected after ITP in a prototype fluidic device.

FIGS. 20A-20C are electropherograms from tests of a pure human DNA sample, mixed human/salmon DNA, and pure salmon DNA subject to a PCR amplification assay.

FIGS. 21A and 21B are images of a detection region from tests run performing ITP in a prototype fluidic device using a trace amount of pure human DNA and mixture of sample containing the trace amount of human DNA with salmon DNA samples.

FIGS. 22A and 22B schematically depict assembly manufacturing techniques in accordance with various embodiments.

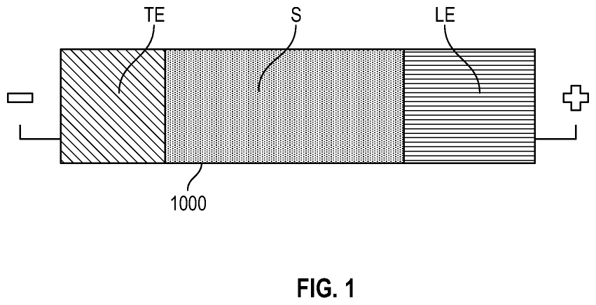

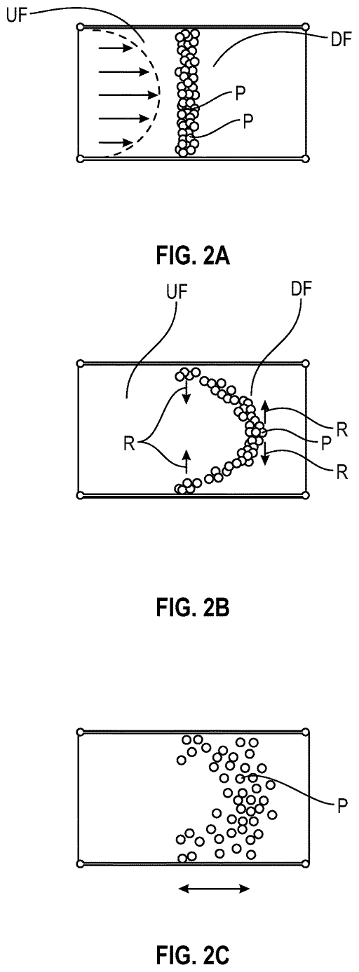

### DETAILED DESCRIPTION

Various embodiments of the present disclosure may demonstrate one or more of the above-mentioned desirable features and/or address the above-mentioned needs. Other features and/or advantages may become apparent from the description that follows.

Additional objects and advantages will be set forth in part in the description which follows, and in part will be obvious from the description, or may be learned by practice of the present teachings. At least some of the objects and advantages of the present disclosure may be realized and attained by means of the elements and combinations particularly pointed out in the appended claims.

It is to be understood that both the foregoing general description and the following detailed description are exemplary and explanatory only and are not restrictive of the present disclosure and claims, including equivalents. It should be understood the present disclosure and claims, in their broadest sense, could be practiced without having one or more features of these exemplary aspects and embodiments. For example, those of ordinary skill in the art would understand that the following detailed description related to various devices, systems, and methods to perform ITP, purification, and enrichment of sample analyte is exemplary only, and that the disclosed devices, systems, and methods can have various components and include other steps that are integrated into part of an overall system for sample processing and analysis, such as for example, various devices, systems, and or methods that are implemented with initial sample preparation and/or analysis via polymerase chain reaction or other nucleic acid amplification and/or sequencing reactions, and/or protein analysis, such as using ELISA assays.

Various embodiments of fluidic devices contemplated by the present disclosure provide a channel used for separation via ITP in a portion of a substrate that is separated from the portion and plane of reservoirs that contain the leading and trailing electrolytes. Through holes that extend through a thickness of the substrate and cross the cross-sectional plane that separates the separation channel from the reservoirs are used to provide flow communication between the reservoirs and separation channel. Such an arrangement can assist with controlled loading of the various substances (e.g., sample, leading electrolyte LE, and trailing electrolyte TE) into the fluidic network while providing controlled hydrostatic pressures that do not cause undesirable bulk fluid flow or introduce air bubbles into the channels. Moreover, the arrangement can allow for a relatively simple workflow in which the fluids (e.g., sample, leading electrolyte LE, and trailing electrolyte TE) can be loaded in a sequential manner into the channel used for ITP separation and without the need to interface the fluids from generally opposing directions of travel for the various fluids being used. Moreover, by providing various embodiments of a fluidic device with different inlet channels in the same portion (plane) of the substrate as the separation channel and in a different portion (plane) than the reservoirs, the hydrostatic pressure can be controlled to alleviate undesirable bulk fluid flow and introduction of air bubbles while still permitting relatively simple loading of the various fluids in a sequential manner without the need to control the interface of the various fluids being used by introducing them in opposing directions of travel. Connections to various fluid sources (e.g., sample, leading electrolyte LE, and trailing electrolyte TE) and supply from those sources also can be simplified and loading automated through appropriate connectors, valve mechanisms, supply sources, and a fluidics controller to control timing of the same and consequent fluid flow. However, the present disclosure also contemplates such loading to take place via manual mechanisms, such as syringes coupled via tubing to ports of the device. Ports that provide fluidic coupling of the inlet channels to fluid supply sources can be either located in the reservoir portion of the substrate or in the channel portion. If located in the latter, the ports can be closed off and dead-ended after introduction of fluid therethrough to control hydrostatic pressure and bulk fluidic flow.

Various embodiments contemplated by the present disclosure provide a viscosity mismatch of the sample and leading electrolyte so as to promote better flow dynamics favorable to ITP. By way of example, the sample containing the ionic analyte can be mixed with an additive reagent that increases the sample mixture viscosity relative to the leading electrolyte such that a more favorable flow profile can be achieved during ITP to mitigate undesirable Taylor diffusion.

Various embodiments contemplated by the present disclosure further enhance enrichment and detection of ionic analyte by utilizing an additive reagent comprising an ionic component that has substantially the same ionic mobility as the ionic analyte. In this way, upon ITP, the ionic component can travel with the ionic analyte in the separation channel and an effect of overall enrichment of the concentration of combined ionic analyte and ionic component in the separated ITP band can be realized, which can allow for detection when the concentration of the ionic analyte alone may make detection difficult. Further, the present disclosure contemplates the use of such additive reagents in which the ionic components do not interfere with further downstream processing or analysis of the ionic analyte after ITP.

### Fluidic Devices and Related Methods of Use

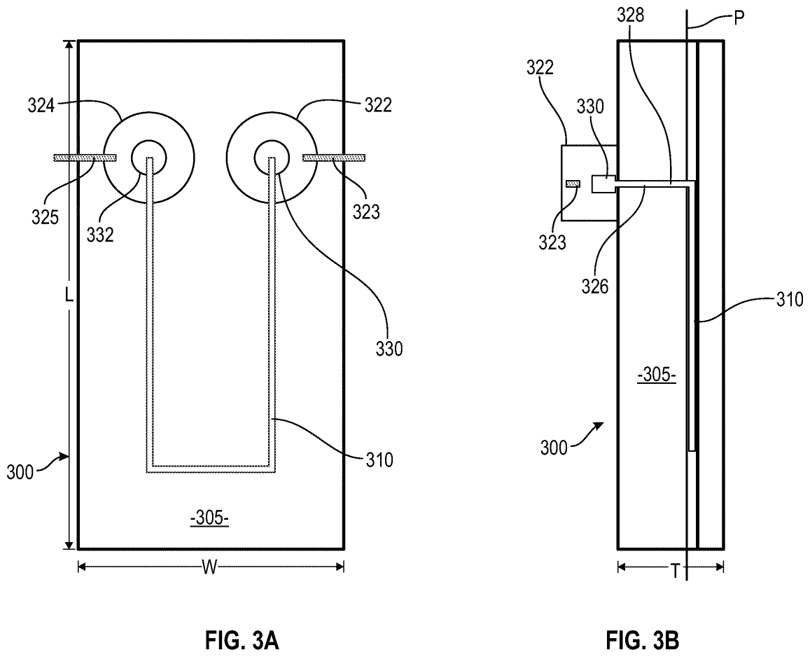

Various embodiments of fluidic devices and operation of the same for implementing ITP to achieve sample purification and enrichment that are contemplated by the present disclosure are illustrated in FIGS. 3A-11G. With initial reference to FIGS. 3A and 3B, showing respectively plan and side views, a fluidic device 300 in accordance with various embodiments comprises a substrate 305 comprising first and second reservoirs 322, 324 in a first portion of the substrate 305 that are fluidically coupled to an intermediate channel 310 fluidically coupled to and extending between the two reservoirs 322, 324. The intermediate channel 310 is in a second portion of the substrate 305, with the first and second portions being separated from one another by a cross-sectional plane P perpendicular to a thickness dimension T of the substrate 305. The plan view of FIG. 3A is from the top of the fluidic device as it would be positioned on a surface while in use, with the opposing side being in contact with the work surface such that gravity acts in the direction into the plane of the illustration. The plan view of FIG. 3A thus shows the channels through interior portions of the substrate (or as projections on the plane of the reservoirs. While the substrate 305 has a thickness dimension, the intermediate channel 310 can be considered to be in a first planar layer of the substrate 305 and the reservoirs 322, 324 in a second planar layer of the substrate. Electrodes 323, 325 are in respective electrically conductive communication with the reservoirs 322, 324. As those of ordinary skill in the art would understand, the reservoirs 322, 324 can be used interchangeably to respectively contain the leading and trailing electrolytes depending on the polarity of the electric field being applied at the electrodes 323, 325 and based on the charge of the ionic analyte of interest, and thus the desired direction of ionic mobility to achieve separation of the ionic components in the sample.

The intermediate channel 310 is fluidically coupled to each of the reservoirs 322, 324 via respective through holes 326, 328 (only one such through hole being visible in the side view of FIG. 3B, with reference label 328 used to point to the through hole that is aligned with and hidden by through hole 326) that extend from the reservoirs 322, 324 at one open end to an opposite open end in fluidic communication with the intermediate 310. The intermediate channel 310 may also be referred to as a separation channel because, as will be understood from further explanation below, the channel 310 is where the separation of ionic components based on ionic mobility after application of an electric field occurs (i.e., where the ITP occurs). As can be seen in the side view of FIG. 3B, the ends of the through holes 326, 328 at the reservoirs 322, 324 are fluidically coupled to ports 330, 332 that extend into the reservoirs 322, 323 and have a height that permits a volume of liquid to be retained therein and such that any flow of fluid from the intermediate channel 310 through the through holes 326, 328 can overflow into and be contained by the reservoirs 322, 324. An open end of the ports 330, 332 is accessible to attach fluid supply conduits and can be configured to permit removably coupling of such supply conduits. In this way pressure (positive or negative) can be delivered to the port, through holes, and separation channels to drive pressure-driven fluid flow.

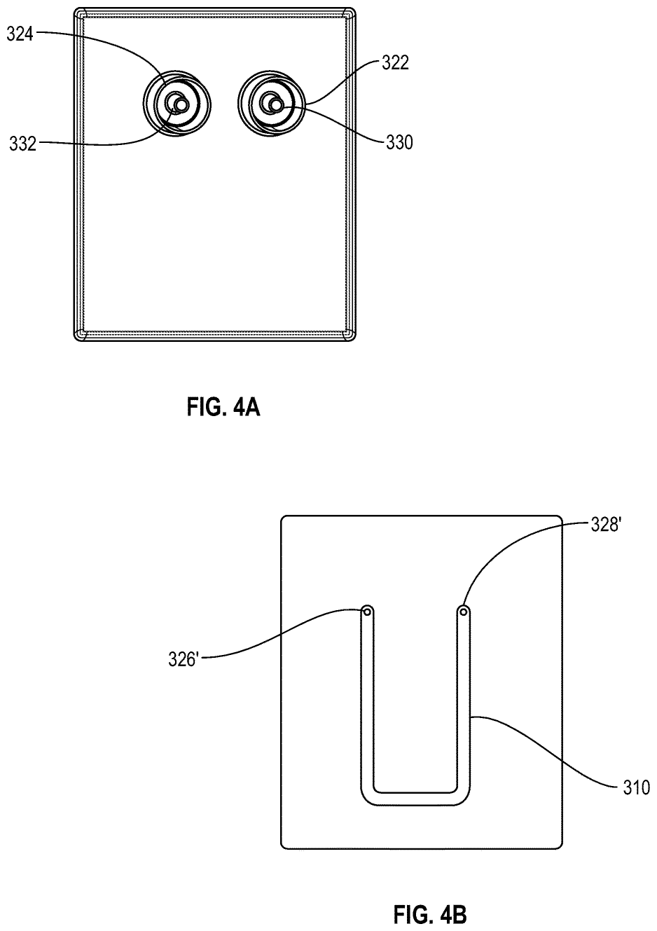

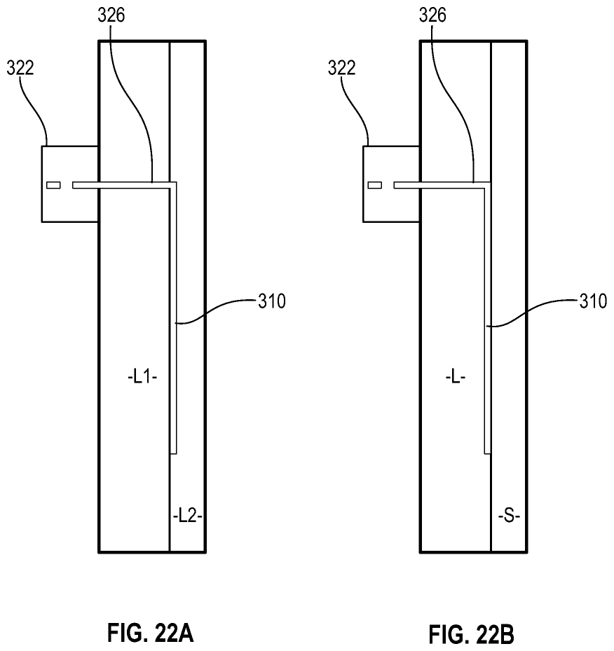

FIGS. 4A and 4B are top perspective views respectively illustrating the first portion of the substrate 305 containing the reservoirs, ports, and through holes (not shown) and the second portion of the substrate 305 comprising the intermediate channel 310 and through holes 326′, 328′which connect to the through holes 326, 328 in the first portion of the substrate 305. Further reference to through holes herein include the entire through hole (e.g., 326 and 326′, 328 and 328′) that is formed through both portions of the substrate 305 and permits flow communication between the ports and intermediate channel 310. In one embodiment, the first portion of FIG. 4A and the second portion of FIG. 4B can be layers that are manufactured separately and assembled together (e.g., as a laminated structure) to form the substrate 305. Reference is made to FIG. 22A schematically showing an embodiment of such a construction in which L1 represents the layer comprising the reservoirs (reservoir 322 being depicted as representative) and through holes (through hole 326 being depicted as representative) and L2 represents the layer comprising the channels (channel 310 being depicted as representative). The layer L2 can be formed with the channels open at the face of the layer that abuts layer L2. In another embodiment, the substrate can be a monolithic structure with the different flow structures (i.e., reservoirs, through holes, and channels) made in each respective portion (plane) of the substrate and a film or other material layer used to seal the open channels in the second portion (plane) of the device. The sealing layer may be, for example, a pressure sensitive adhesive film or bonded film assisted by heat, solvents, and/or pressure. Reference is made to FIG. 22B schematically showing an embodiment of such a construction in which layer L is a monolithic structure formed with the reservoirs, through holes, and channels (representative reservoir 322, through hole 326, and channel 310 being shown) lying in respective portions (planes) of the monolithic layer L and with the channels (310 being shown) open to the face of the layer L that faces opposite to the face containing the reservoirs. In FIG. 22B, a film or other sealing material is a layer S placed over the face of the open channels to seal the channels. In yet a further embodiment, a single monolithic structure could be used with the channels and through holes formed within the thickness of the structure, such as via laser ablation, 3d-printing or other additive manufacturing techniques, for example. In the assembling manufacturing techniques described above with reference to FIGS. 22A and 22B, the various fluidic structures may be made via injection molding, hot embossing, wet etching and other similar techniques as those of ordinary skill in the art would be familiar with.

The configuration of the reservoirs, through holes, and intermediate channel of the embodiment of FIGS. 3 and 4 assists with maintaining hydrostatic pressure in the fluidic network by permitting the through holes 326, 328 via the ports 330, 332, to provide venting to the atmosphere and with the level of the liquid in the reservoirs below the opening of the through hole in the reservoir. In addition, in a state of a reservoir 322, 324 being filled with liquid (e.g., leading or trailing electrolyte) to a level above the opening of the ports 330, 332 extending into the reservoirs 322, 324, the pressure head in the reservoirs 322, 324 can be such as to maintain hydrostatic pressure at a desired level and provide resistance to any bulk fluid flow in the intermediate channel 310 that may not be desirable. In other words, the configuration of the reservoirs in one planar layer of the substrate and the intermediate channel (to be used as a separation channel for ITP) in another can permit pressure heads to be utilized via filling the reservoirs that maintain stability in the hydrostatic pressure experienced in the channel network (i.e., separation channel 310 of the embodiment of FIGS. 3 and 4) and prevent undesirable bulk fluid flow disturbances. Moreover, as will be better understood by the description which follows regarding loading and operation of the fluidic device to perform ITP, the configuration and arrangement of the reservoirs, ports/through holes, and intermediate channel promotes a relatively straightforward loading of the channel 310 without trapping air bubbles, thus enhancing the ability to effectively create a boundary and interface between the sample and the leading and trailing electrolytes, respectively.

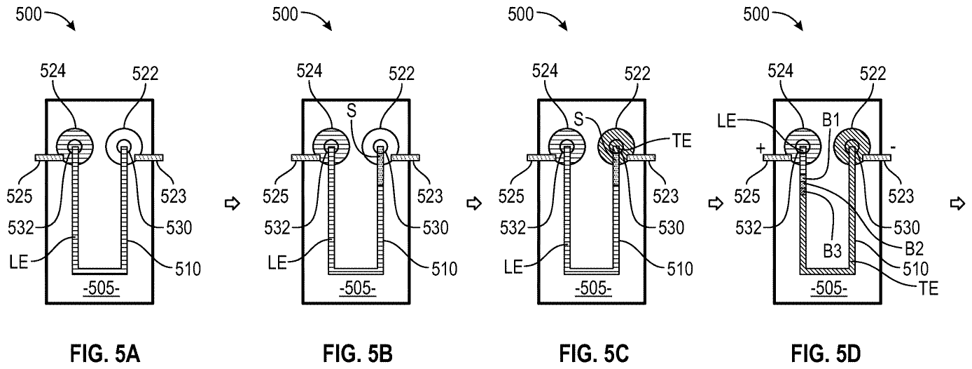

Turning now to FIGS. 5A-5D, a schematic depiction of an embodiment of a workflow for use of the fluidic device embodiment of FIGS. 3-4 for purification and enrichment of a negatively charged ionic analyte in a sample using ITP will be described. Different shadings are used to represent the different substances being loaded into the device. In a first stage, depicted by FIG. 5A, the intermediate channel 310 and the reservoir 324 are primed with leading electrolyte LE. This can be accomplished by loading the leading electrolyte LE through port 332 until it fills reservoir 324 via application of a positive pressure delivery mechanism (e.g., a syringe or other pumping mechanisms) or by filling the reservoir 324 and drawing the leading electrolyte LE into the port 332, through hole 328 and through the intermediate channel 310 by a negative pressure (vacuum) at port 330. Other techniques to prime the intermediate channel 310 and fill the reservoir 324 and port 330 with the leading electrolyte LE would be understood by those of ordinary skill in the art. After priming the reservoir 324 and intermediate channel 310 with leading electrolyte LE, the intermediate channel 310 can be primed with a volume of sample S to be analyzed by introducing the volume of sample S through the port 330 and through hole 326, as depicted in FIG. 5B.

Once the volume of sample S to be analyzed has been primed in the intermediate channel 310, the trailing electrolyte TE can be primed into the reservoir 322 and the port 330, as shown in FIG. 5C. Finally, as illustrated in FIG. 5D, once the device has been loaded as depicted, the electric field can be applied by activating the electrodes 323, 325. FIG. 5D illustrates a scenario in which the electrode 325 is the anode and electrode 323 the cathode, thereby being suitable for purification of a sample in which the ionic analyte is negatively charged. As shown in FIG. 5D, after a sufficient period of time has elapsed, the leading ions of the leading electrolyte LE, ionic analyte, and ionic impurities of the sample, and trailing ions of the trailing electrolyte TE can move through the intermediate channel 310 with the ionic analyte and ionic impurities of the sample eventually become separated from each other and forming distinct bands (labeled B1, B2, B3) based on the ionic mobility of each. The banding can therefore allow for further detection, collection, and/or processing (e.g. analysis) of each ionic species as desired and as would be understood by those of ordinary skill in the art.

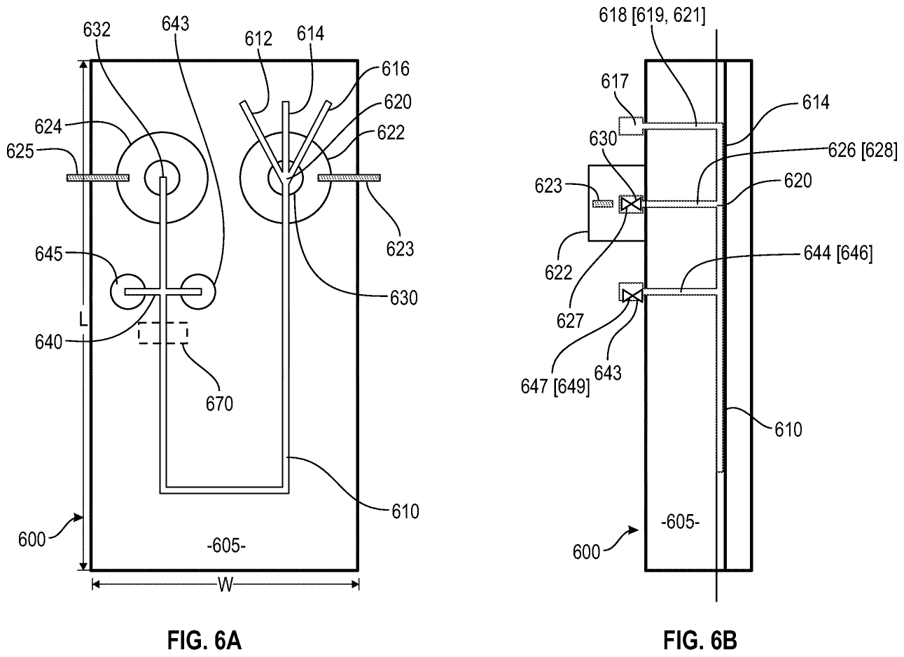

The present disclosure further contemplates embodiments of fluidic devices that include one or more channels in addition to the intermediate channel connecting the two reservoirs for leading and trailing electrolyte and which serves as the channel to allow for the separation of ionic components when using ITP. Such additional channels can be one or more inlet channels used to supply one or more of the sample, trailing electrolyte TE, and leading electrolyte LE, or one or more outlet channels to collect substances from the fluidic device, such as one or more ionic analytes or waste. Various embodiments with such additional channels are explained further below with reference to FIGS. 6A-11G. Those having ordinary skill in the art would appreciate that other embodiments (not shown) can include more or fewer of the additional channels in varying configurations within the device without departing from the scope of the present disclosure.

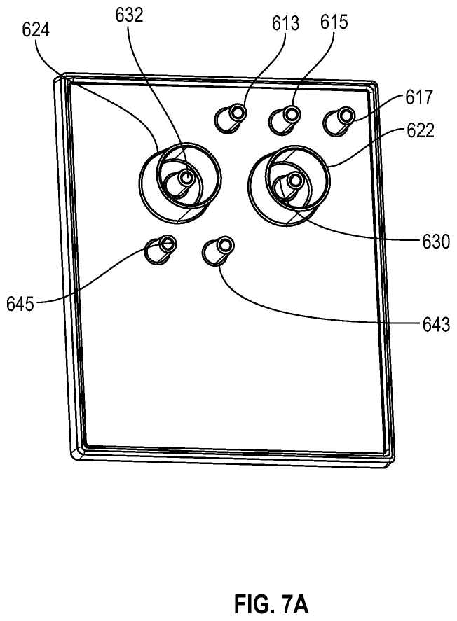

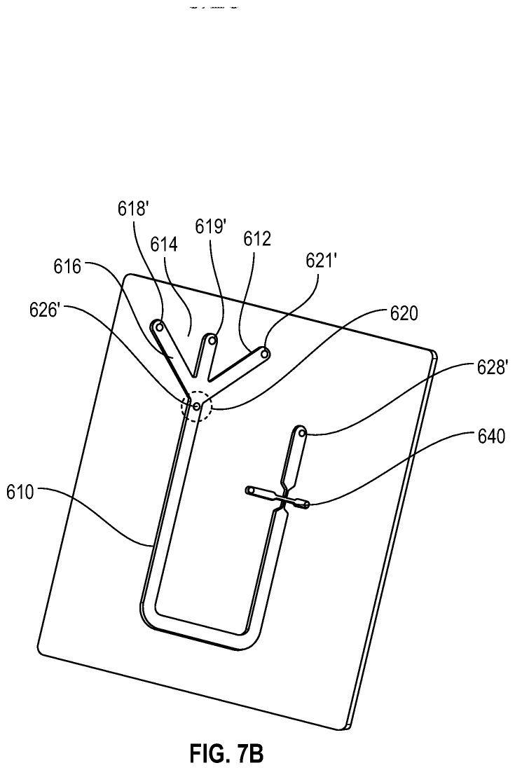

FIGS. 6A-8G illustrate another embodiment of a fluidic device and an embodiment of a workflow for use of the fluidic device. FIGS. 6A and 6B respectively illustrate plan and side views of the fluidic device 600, and FIGS. 7A and 7B illustrate perspective views of a first portion containing reservoirs, ports, and through holes and a second portion containing channels, similar to the views of FIGS. 3A-4B described above. Elements of the fluidic device 600 in the embodiment of FIGS. 6A-8G that are similar to elements in the fluidic device 300 described above are labeled with similar reference numerals except with a 600 series rather than a 300 series. Similar elements and their respective functions and/or alternative configurations and/or arrangements will not be described again here to avoid redundancy and simplify the description. Moreover, although not described with reference to the embodiment of FIGS. 3A-5D, those having ordinary skill in the art would understand that the detection and/or collection channels described below embodiment could be used with the embodiment of the fluidic device of FIGS. 3A-5D.

In the embodiment of FIGS. 6A-7B, the fluidic device 600 comprises inlet channels 612, 614, 616 that are fluidically coupled to respective inlet ports 613, 615, 617 at one end via respective through holes (only one of which 618 is visible and the others 619, 621 being labeled in brackets and not visible because they are hidden from view in FIG. 6B) and meet a juncture 620 (indicated by the dashed circle in the figure) at which they are fluidically coupled to the intermediate channel 610 (via the extension of the through holes 621′, 619′, 618′) at another end. The juncture 620 at which the inlet channels 612, 614, 616 meet the intermediate channel 610 is also fluidically coupled to the reservoir 622 via the through hole 626 (including through hole portion 626′) and port 630. In the fluidic device 600, a valve 627 (shown in FIG. 6B) is provided to selectively open and close the fluidic communication between the port 630 and reservoir 622. As with the embodiment of FIGS. 3A-5D, reference to the through holes of the fluidic device 600 includes the entireties of the through holes (i.e., 626 and 626′, 618 and 618′, 619 and 619′, and 621 and 621′).

In addition to the inlet channels, inlet ports, and their respective through holes, the fluidic device 600 further comprises a collection channel 640 that intersects the intermediate channel 610. The location at which the collection channel 640 intersects the channel 610 is sufficiently downstream of the reservoir 622 so that enough time and overall length of the channel 610 can permit the desired ionic motion and separation into sufficiently distinct bands of the ionic components of interest when performing ITP in the device 600. In various embodiments, the collection channel 640 can be placed at a location along the overall length of the intermediate channel 610 such that a ratio of the length of the intermediate channel 610 from the reservoir 622 to the location of the collection channel 640 to the overall length of the intermediate channel 610 between the reservoirs 622 and 624 is in a range of from at least 0.7 to 1.0. Although the collection channel 640 is depicted at a location upstream of the reservoir 624 in the embodiment depicted in FIGS. 6A-7B, those of ordinary skill in the art would appreciate that the collection channel could be placed downstream of the reservoir 624 without departing from the scope of the present disclosure. The collection channel 640 is fluidically connected to ports 643, 645 at each of its ends via through holes (with only one of the through holes 644 being visible in FIG. 6B and the other 646 being labeled in brackets and hidden from view). Upstream of collection channel 640 is a detection region 670, which can comprise any of a variety of detection mechanisms to detect one or more of the ionic components migrating through the intermediate channel 610. Such detection mechanisms can be integral with a portion of the substrate 605 or may be external to the substrate 605 and otherwise part of an overall system comprising the fluidic device 600. Those having ordinary skill in the art would appreciate a variety of ways in which such a detection mechanism can be incorporated.

The detection mechanism can be any of a variety of detection mechanisms, such as, for example, such as a variety of optical detection mechanisms that can detect colorimetric and/or fluorescence. In an embodiment, an optical fiber may be used to emit electromagnetic radiation at a predetermined wavelength toward a transparent region of the intermediate channel 610 at the detection region 670. A spectrometer can be used to detect electromagnetic radiation emitted substances passing through the detection region 670. A controller operably coupled to the spectrometer can be programmed to trigger collection through the collection channel 640 based on sensing one or more predetermined wavelengths of electromagnetic radiation emitted through the separation channel 610 at the detection region 670. The controller can be operably coupled to control valves associated with ports 643 and 645 (one such valve 647 being shown in FIG. 6B and the other valve 649 labeled in brackets and hidden from view in FIG. 6B), which can be triggered to open flow through the collection channel 640 based on the detected electromagnetic radiation. In this way, by knowledge of the relative ionic mobilities of the differing components (e.g., leading electrolyte, ionic analyte, ionic impurities, trailing electrolyte) and the emission coming from the differing components separating into the differing and distinct bands as they travel via ITP through the intermediate channel 610, the detection mechanism can detect the bands as they travel through detection region 670 and trigger the collection through the collection channel 640 and ports 643, 645 (e.g., one for ionic analytes and one for waste/impurities).

Detection mechanisms used to provide detection of the bands may include, for example, optical or electrical detection mechanisms. Optical detection mechanisms can directly measure particular wavelength absorbance of analytes, or via tagging analyte with fluorescent dye through covalent bonding, intercalating, and/or adsorption. Electrical detection mechanisms can detect the change of the impedance, conductivity, and/or resistivity of the fluids. In cases in which an unknown number of impurities with relatively higher ionic mobility may exist, optical detection may be a more reliable detection technique. Those having ordinary skill in the art would have familiarity with various such detection mechanisms and understand how to choose appropriate detection mechanisms based on particular applications.

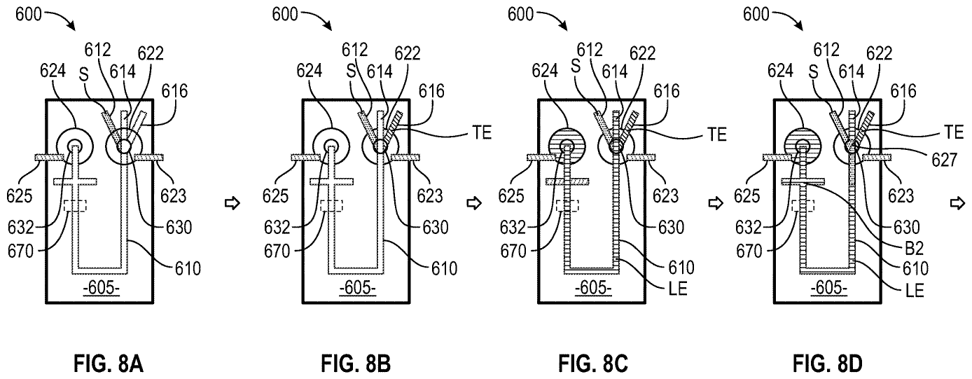

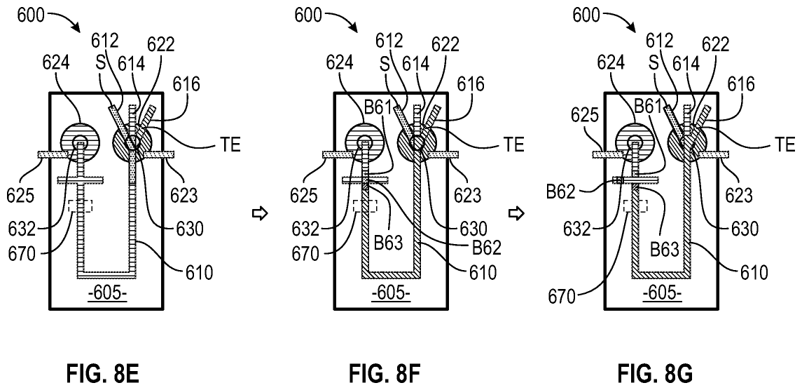

Turning now to FIGS. 8A-8G, a schematic depiction of an embodiment of a workflow for use of the fluidic device 600 for purification and enrichment via ITP of a negatively charged ionic analyte in a sample will be described. Different shadings are used to represent the different substances being loaded into the device. In a first stage, depicted by FIG. 8A, inlet channel 612 is primed with the sample S containing ionic analyte and impurities. In FIG. 8B, the inlet channel 616 is primed with trailing electrolyte TE. When priming the sample S in inlet channel 612 and trailing electrolyte TE in inlet channel 616, a predetermined volume can be used to fill the inlet channels. The valve 627 can be in an open state in such a situation. After inlet channels 612, 616 have been primed with sample S and trailing electrolyte TE, respectively, valve 627 can be closed (indicated by small diagonal line in FIG. 8C) and leading electrolyte LE can be introduced through inlet channel 614 in a volume sufficient to fill (prime) inlet channel 614 and intermediate channel 610 and at least partially fill reservoir 624, as shown in FIG. 8C.

In FIG. 8D, once the leading electrolyte LE has at least partially filled the reservoir 624 and filled intermediate channel 610 and inlet channel 614, LE is primed and fills collection channel 640. Additional sample S or other positive pressure can be applied to inlet channel 612 to flow a volume of sample S into intermediate channel 610 and into contact with leading electrolyte LE. Because valve 627 is in a closed state, the path of least resistance for the sample S is into the intermediate channel 610. Further, because the leading electrolyte LE fills separation channel 610, the juncture 620 and inlet channel 614, and the reservoir 624, trapping air bubbles should be significantly reduced or prevented when loading the sample S from inlet channel 612 into the separation channel 610. Moreover, any displacement of leading electrolyte LE due to loading sample S into the intermediate channel 610 can overflow through port 632, via its corresponding through hole 628, into reservoir 624. With reference to FIG. 8E, after sample S has been loaded into intermediate channel 610, valve 627 can be placed in an open state to place reservoir 622 into fluidic communication with port 630. Once valve 627 is opened, additional trailing electrolyte TE can be added through inlet channel 616 or pressure otherwise applied to inlet channel 616. The path of least resistance in the state of the fluidic network in FIG. 8E for the trailing electrolyte TE is from inlet channel 616 through hole 626, port 630, and into reservoir 622. Thus, trailing electrolyte TE can at least partially fill reservoir 622 while inlet channel 616 also remains primed with trailing electrolyte TE.

FIG. 8E represents the state of the fluidic device 600 in a fully loaded configuration and ready to perform ITP. At this stage, and as reflected in FIG. 8F, a voltage can be applied to electrodes 623, 625 to create an electric potential and an electric field across the intermediate channel 610 at a voltage level and time period sufficient for the various ions to separate and band together based on their respective ionic mobilities. As with the embodiment in FIGS. 5A-5D, FIG. 8E illustrates a scenario in which the electrode 625 is the anode and electrode 623 is the cathode, thereby being suitable for purification of a sample S in which the ionic analyte is negatively charged. As shown in FIG. 8E, after a sufficient period of time as elapsed, the ionic analyte, leading ions of the leading electrolyte LE, and ionic impurities of the sample S, and trailing ions can move through the separation channel 610 and into distinct bands (labeled B61, B62, B63) based on the ionic mobility of each.

Utilizing a detection mechanism, as discussed above with reference to FIG. 6A, as further illustrated in FIG. 8G, upon detection of the ionic analyte (labeled B62), the high voltage across the intermediate channel 610 is terminated and collection through collection channel 640 and port 645 can be triggered. In one embodiment, collection can occur hydrodynamically, for example by opening valve 649 and applying an appropriate force such as a suction force at port 645 and/or a positive pressure at valve 643. In another embodiment, collection can occur electrically by utilizing a secondary anode (not shown) in port 645 and creating a potential difference to cause the ionic analyte to move to through the port 645. In an embodiment, other of the separated and banded ionic components (e.g., B61, B63) may be further flowed through the collection channel 640. For example, if more than one ionic analyte of interest is present, it may be possible to collect each through the two different ends of channel 640 and corresponding ports 643, 645 and valves 647, 649. Alternatively, impurities may be collected through one port 643, 645 and the ionic analyte of interest through the other port 643, 645. The ionic components of the leading and trailing electrolytes LE, TE can be flowed to the reservoir 624. Those of ordinary skill in the art would appreciate various ways in which collection of the ionic components B61, B62, B63 can be detected and collected.

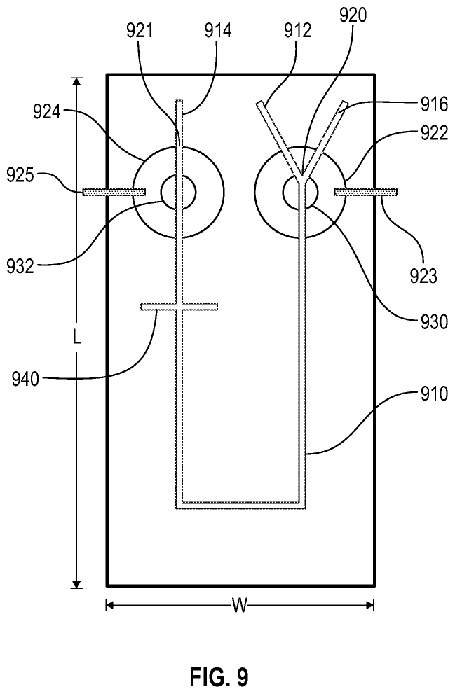

Another embodiment of a fluidic device and workflow for use of the device to perform sample purification and enrichment is illustrated in FIGS. 9A-11G. The fluidic device 900 and its operation is similar to the fluidic device 600 as described above with reference to FIGS. 6A-8G, with the exception of the configuration of the inlet channels. Similar parts are thus labeled with similar reference numerals except using a 900 series instead of the 600 series used in the embodiments of FIGS. 6A-8G. With the exception of the description of the arrangement of inlet channels, the remainder of the parts and the views are as in FIGS. 6A-8G and the description thereof will not be repeated here so as to avoid redundancy. The fluidic device 900 comprises a substrate 905 having two inlet channels 912 and 916 fluidically coupled to inlet ports 913 and 917 via through holes 921, 918 at one end and to the intermediate channel 910 the other ends, where they meet with each other and the separation channel 910 at a juncture 920. Another inlet channel 914 is coupled to the separation channel 910 at a juncture 950 at a location where the port 932 is fluidically coupled to the intermediate channel 910. The other end of the inlet channel 914 is fluidically coupled to port 915.

As discussed above with regard to an embodiment of a workflow using the fluidic device 600, providing the inlet channels 912, 914, and 916 in the same portion and plane of the substrate 905 as the intermediate channel 910 and separated from the portion and plane of the reservoirs 922, 924 and ports 930, 932 can assist with controlled loading of the various substances (e.g., sample, leading electrolyte LE, and trailing electrolyte TE) into the fluidic network, via the use of the through holes, while providing controlled hydrostatic pressures that do not cause undesirable bulk fluid flow or introduce air bubbles into the channels. Moreover, the arrangement can allow for a relatively simple workflow in which the fluids (e.g., sample, leading electrolyte LE, and trailing electrolyte TE) can be loaded in a sequential manner into the intermediate channel 910. Moreover, by providing the fluidic device 900 with different inlet ports and inlet channels, connections to various fluid sources (e.g., sample, leading electrolyte LE, and trailing electrolyte TE) and supply from those sources can be simplified and loading automated through appropriate connectors, valve mechanisms, supply sources, and a fluidics controller to control timing of the same and consequent fluid flow. However, the present disclosure also contemplates such loading to take place via manual mechanisms as well, such as syringes coupled via tubing to the ports etc.

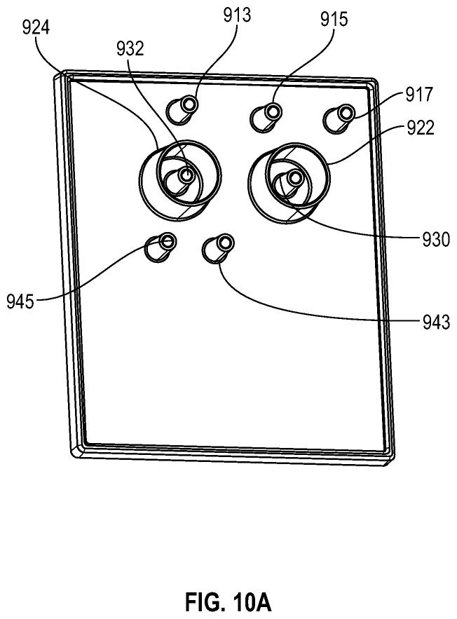

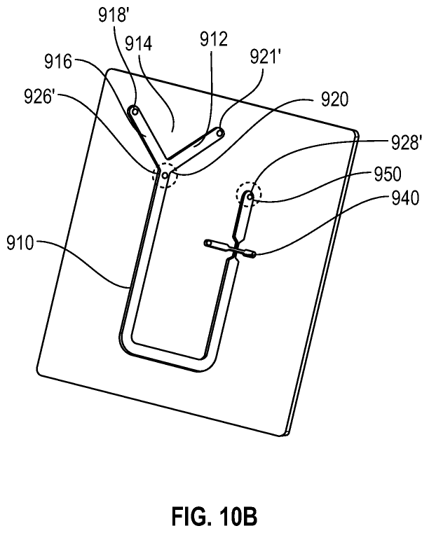

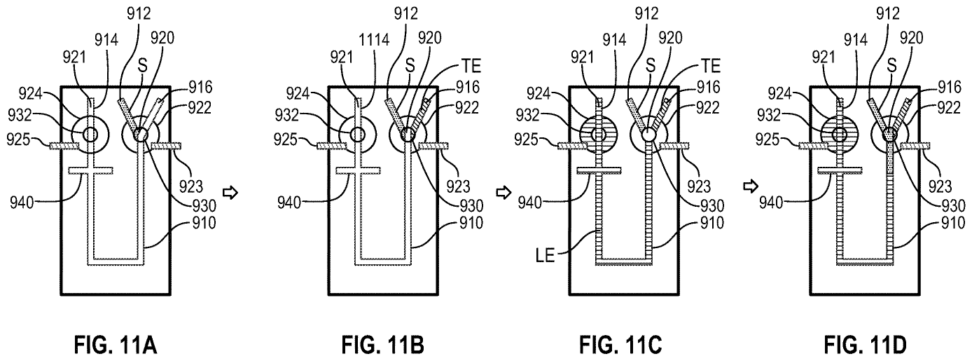

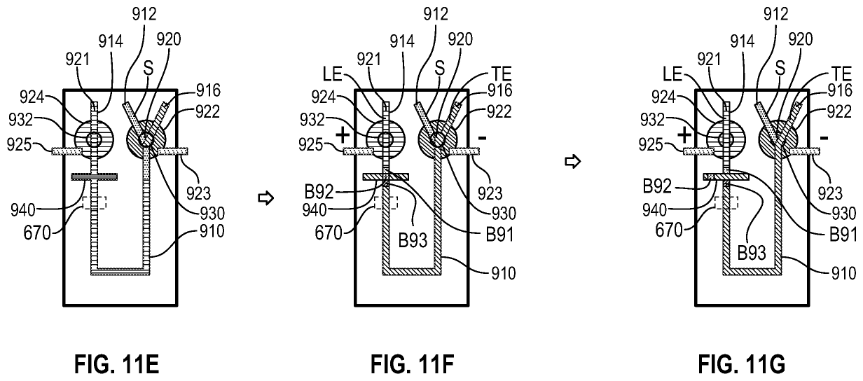

A schematic depiction of an embodiment of a workflow for use of the fluidic device 900 for purification and enrichment via ITP of a negatively charged ionic analyte in a sample is illustrated in FIGS. 11A-11G. Different shadings are used to represent the different substances being loaded into the device. In a first stage, depicted by FIG. 11A, inlet channel 912 is primed with the sample S containing ionic analyte and impurities and inlet channel 916 can be primed with trailing electrolyte TE, as shown in FIG. 11B. Those having ordinary skill in the art would understand that the sample S in inlet channel 912 and the trailing electrolyte TE in inlet channel 916 can be primed simultaneously or in opposite order as shown in FIGS. 11A and 11B. When priming the sample S in inlet channel 912 and trailing electrolyte TE in inlet channel 916, a predetermined volume can be used to fill the respective inlet channels 912, 916. The valve 927 can be in an open state in such a situation. After inlet channels 912, 916 have been respectively primed with sample S and trailing electrolyte TE, and with valve 927 in an open state, leading electrolyte LE can be introduced through inlet channel 914 in a volume sufficient to fill (prime) inlet channel 914, intermediate channel 910, and at least partially fill reservoir 924, as shown in FIG. 11C. Because of the pressure heads created in the primed inlet channels 912, 916, once the intermediate channel 910 has been filled with leading electrolyte LE, additional leading electrolyte LE will flow through the port 932 and corresponding through hole 928 (portion 928′being shown in FIG. 10B), into reservoir 924 due to the exposure to ambient pressure and that being the path of least resistance. Once the leading electrolyte LE has at least partially filled the reservoir 924 and filled intermediate channel 910 and inlet channel 914, LE is primed and fills collection channel 940. In FIG. 11D, once the leading electrolyte LE has at least partially filled the reservoir 924 and filled separation channel 910 and inlet channel 914, and collection channel 940, valve 927 associated with port 930 and its corresponding through hole (portion 926′shown in FIG. 10B) can be closed and additional sample S or other positive pressure can be applied to inlet channel 912 to flow a volume of sample S into intermediate channel 910 and into contact with leading electrolyte LE. Because valve 927 is in a closed state, the path of least resistance for the sample S is into intermediate channel 910. Further, because the leading electrolyte LE fills intermediate channel 910, the junctures 920, 950, and the reservoir 924, trapping of air bubbles can be substantially minimized or prevented when loading the sample S from inlet channel 912 into the intermediate channel 910. Moreover, any displacement of leading electrolyte LE due to loading sample S into the intermediate channel 910 can overflow through port 932, via its corresponding through hole 928, into reservoir 924. With reference to FIG. 11E, after sample S has been loaded into intermediate channel 910, valve 927 can be placed in an open state to place the through hole 926 corresponding to port 930 into fluidic communication with port 930. Once valve 927 is opened, additional trailing electrolyte TE can be added through inlet channel 916 or pressure otherwise applied to inlet channel 916. The path of least resistance in the state of the fluidic network in FIG. 11E for the trailing electrolyte TE is from inlet channel 916 through the through hole and corresponding port 930, and into reservoir 922. Thus, trailing electrolyte TE can at least partially fill reservoir 922 while inlet channel 916 and juncture 920 also remains primed with trailing electrolyte TE.

FIG. 11E represents the state of the fluidic device 900 in a fully loaded configuration and ready to perform ITP. At this stage, and as reflected in FIG. 11F, a voltage can be applied to electrodes 923, 925 to create an electric potential and an electric field across the intermediate channel 910 at a voltage level and over a time period sufficient for separation and banding of the various ionic components to occur. FIG. 11E illustrates a scenario in which the electrode 925 is the anode and electrode 923 is the cathode, thereby being suitable for purification of a sample S in which the ionic analyte is negatively charged. As shown in FIG. 11E, after a sufficient period of time as elapsed, the ionic analyte, leading ions of the leading electrolyte LE, and ionic impurities of the sample S, and trailing ions can move through the separation channel 910 and into distinct bands (labeled B91, B92, B93) based on the ionic mobility of each.

Utilizing a detection mechanism, as discussed above with reference to the embodiment of FIGS. 6A-8G, as further illustrated in FIG. 11G, upon detection of the ionic analyte (labeled B92), collection through collection channel 940 and port 945 can be triggered, in a manner similar to that described above with reference to the embodiment of FIGS. 8A-8G. In an embodiment, other of the separated and banded ionic components (e.g., B91, B93) may be further flowed through the collection channel 940. For example, if more than one ionic analyte of interest is present, it may be possible to collect each through the two different ends of channel 940 and corresponding ports 943, 945. Alternatively, impurities may be collected through one port 943, 945 and the ionic analyte of interest through the other port 943, 945. The ionic components of the leading and trailing electrolytes LE, TE can be flowed to the reservoir 924. Those of ordinary skill in the art would appreciate various ways in which collection of the ionic components B91, B92, B93 can be detected and collected.

In the above descriptions of the workflows of FIGS. 8A-8G and 11A-11G, it should be understood that loading through the various inlet channels occurs through the various ports associated with those inlet channels even if not explicitly described.

While various embodiments illustrated herein have a U-shaped intermediate channel, the intermediate channel is not limited to such a configuration and can have a variety of shapes between the leading and trailing electrolyte reservoirs, such as but not limited to, a serpentine pattern, a V-shape, an S-shape, or any other paths providing an overall length of the channel between the electrolyte channels sufficient to allow for enough ionic mobility and separation to occur and create distinct bands of ionic components. Those having ordinary skill in the art would understand how to select an overall length and shape of the separation channel depending on factors such as the volume of the separation channel, the ionic analyte of interest, the trailing and leading electrolytes used, the strength of the electric field, the concentration of the ionic analyte in the sample, the center-to-center distance along a width dimension and/or the length dimension of the substrate of the reservoirs. Similarly, the location at which collection occurs (e.g., via a collection channel) along the length of the intermediate channel may be similarly chosen so as to be at a location along the length of the intermediate channel or downstream of such a location such that a sufficient length of the channel to achieve the desired distinct bands and separation of ionic components occurs prior to collection.

In various embodiments, fluidic devices in accordance with the present disclosure, such as fluidic devices 300, 600, and 900 have dimensions and arrangements of the various components selected so as to provide additional mitigation of bubble formation and/or favorable hydraulic pressure conditions. For example, an overall length to width aspect ratio of the substrate (denoted by L and W in FIGS. 3A, 6A, and 9A, may be in a range of about 0.7 and 1.4. Further, the distance between the through holes corresponding to the ports of the reservoirs (such as reservoirs 322/324, 622/624, and 922/924) (e.g., the center-to-center distance of the reservoirs) along the width dimension W of the fluidic devices may, in various embodiments, range from 5 mm to 50 mm, and along the length dimension L may range from 5 mm to 50 mm. Those of ordinary skill in the art would appreciate that the various dimensions can impact the degree of tilt and/or nonuniformities of surfaces of the substrate that can be accommodated while still maintaining desired pressure heads to achieve controlled loading and desirable hydrodynamic conditions. In various embodiments, the intermediate channel extending between the reservoirs can also stay within the same overall width as the distance between the through holes associated with the electrolyte reservoirs.

The valves that may be used to selectively open and close the through holes and/or ports of the fluidic devices in accordance with various embodiments described can be a solenoid pinch valve. Those having ordinary skill in the art would appreciate a variety of types of controllable one-way valve mechanisms that may be used without departing from the scope of the present disclosure.

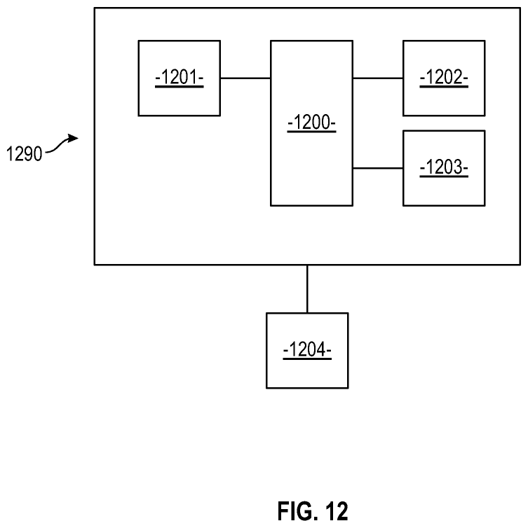

The ports of fluidic devices in accordance with various embodiments of fluidic devices (such as fluidic devices 300, 600, and 900) may be configured to be fluidically coupled with tubing (such as silicone tubing) and syringes for application of pressure (positive or negative) to load and withdraw fluids from the fluidic device. In other embodiments, it is contemplated that the fluidic device is part of an overall integrated system and the ports at which fluids are loaded and withdrawn (collected) from the fluid device is via other types of fluidic couplings as those having ordinary skill in the art would be familiar with. In a non-limiting embodiment, an overall system may be such as illustrated in the block diagram of FIG. 12. In the embodiment of FIG. 12, a system 1290 for sample processing and analysis may include a sample and reagent supply module 1201, configured to provide one or more sources of the sample and reagents (e.g., leading electrolyte, trailing electrolyte, etc.) used to perform ITP. The sample and reagent supply module 1201 can be fluidically coupled to a fluidic device 1200 configured to perform ITP, such as any of the fluidic device 300, 600, 900 described above. Appropriate reservoirs to individually contain the fluids to be supplied to fluidic device and appropriate fluidic couplings and flow control devices (e.g., valves) can be connected via the ports of the fluidic device to load and withdraw substances from the fluidic device 1200, for example, in accordance with the various embodiments of workflows disclosed herein. In an embodiment, the supply module 1201 may further include a lysis, filtration, or other sample preprocessing mechanism prior to introducing the sample containing ionic analyte and impurities to the fluidic device 1200. The system 1290 may also include a downstream analysis module 1202 that may be coupled to the fluidic device 1200, such as to one or more collection ports (such as collection ports 343 and/or 345) through appropriate fluidic couplings and flow control devices. In an embodiment, the downstream analysis module 1202 may provide a source of negative pressure (suction) to withdraw substances from the fluidic device and/or may include an electrical interface to withdraw charged substances via electrical potential. In an embodiment the downstream analysis module may be configured to perform an amplification assay, such as, but not limited to polymerase chain reaction (PCR) and detection. A variety of fluidic (chip-based) PCR analysis platforms are known to those of ordinary skill in the art. Other applications for which fluidic devices of the present disclosure may be used include, but are not limited to, protein purification, including for proteomics and drug development. Detection methods for such applications can include optical detection techniques based on measurements of absorbance spectrum or via fluorescent labeling via antibodies. The system 1290 may further include a waste module 1203 to withdraw, such as via one of the collection ports (e.g., one or both of 343, 345; 643, 645; 943, 945) from the fluidic device and to contain and/or further process the same. A control module 1204 can also be part of the system 1290 and include processing capabilities so as to allow programmed instructions to be stored to control over timing and operation of fluids to be supplied and/or collected from the fluidic device 1200, including control over priming of the channels and reservoirs, the opening and closing of valves, the application of pressure (positive or negative), and the timing and voltage levels of the electrodes in a manner so as to utilize the fluidic device for ITP, such as, for example, in accordance with the exemplary workflows described above. Those having ordinary skill in the art would be familiar with a variety of different components that could be integrated to achieve an overall sample preparation, processing, and analysis, in combination with the fluidic devices contemplated herein for performing sample purification and enrichment using ITP. Those of ordinary skill in the art would also understand that a system that integrates the fluidic devices of the present disclosure need not include all of the components shown in the embodiment of system 1290.

In accordance with various embodiments, fluidic devices (such as fluidic device 300, 600, 900) can be made using a variety of techniques familiar to those having ordinary skill in the art, including but not limited to additive manufacturing such as 3D printing, soft lithography, hot embossing, wet etching, laser ablation, and/or injection molding, and using the various techniques and assembly methods described with reference to FIGS. 22A and 22B. Materials to make the fluidic devices can include, but are not limited to,

[symbol]

(PDMS), cyclic olefin co-polymer (COC), glass, metal.

### Additive Reagents

To further facilitate and enhance sample purification and enrichment using ITP, embodiments of the present disclosure contemplate the use of various additive reagents to the sample. In some embodiments, an additive reagent that promotes flow dynamics in the channel in which ITP occurs so as to avoid the Taylor diffusion phenomenon may be used. In some embodiments, an additive reagent that promotes detection (and thus enrichment) of relatively small concentrations of separated and banded ionic analyte resulting from ITP may be used. Such an additive reagent may be used as an additive to the sample being processed so as to enhance enrichment and detection of an ionic analyte, which may be beneficial when small sample volume and resulting smaller concentrations of ionic analyte exist even after purification and banding of the ionic analyte has occurred such that detection is difficult.

In various embodiments, to promote flow dynamics that minimize or avoid the Taylor diffusion phenomenon, an additive reagent can be mixed with the sample so as to alter the viscosity of the sample such that it is higher than that of the leading electrolyte and/or trailing electrolyte. The difference in viscosity can range from 1 to 10 centipoise (cp). In various embodiments, the viscosity of the resulting mixture of the sample and additive reagent can range from 2 centipoise to 10 centipoise. Additive reagents that can be used to increase the viscosity of the sample to provide favorable flow dynamics to minimize Taylor diffusion effects include, but are not limited to, for example, polydimethylacrylate (PDMA), Ficoll, Dextran, and combinations thereof. Suitable additive reagents to increase the viscosity may be selected so as to be water soluble and non-interfering with ITP, such as various water soluble polymers that are neutrally charged. In some embodiments, the viscosity-increasing reagent additive can be mixed directly with the sample being introduced to the fluidic device, such as with a cell lysate for example. In other embodiments, the viscosity-increasing additive reagent can be mixed as part of the pre-processing of the sample prior to introduction to the fluidic device, such as being mixed with a lysis buffer, such as for example, in module 1201 in the system 1290.

In other embodiments, the present disclosure contemplates utilizing an additive reagent mixed with the sample to facilitate enrichment and detection of the ionic analyte. In some circumstances, when the ionic analyte, even after being separated from impurities and aggregated to its discrete band within a separation channel during ITP, has concentration that is low enough to make detection difficult, a reagent additive that has an ionic mobility that is substantially the same as the ionic mobility of the ionic analyte can be mixed with the sample. By using such a reagent additive, a band of higher concentration of ions having the same (or substantially the same) ionic mobility can occur. The greater concentration of such ions includes ions of the analyte of interest and ions of the additive reagent. By increasing the concentration of ions in the band containing the ionic analyte, enrichment in the band of detectable ions can occur. In various embodiments, the additive reagent that contains ions of an ionic mobility substantially the same as that of the ionic analyte can be further selected so as to not interfere with downstream processing of the sample analyte, or it can be otherwise selected so as to allow for further separation or filtering of the same from the sample analyte. In various embodiments, the ionic mobility of the ionic analyte and the ionic mobility of the ionic component are within a range of +/−5% of each other.

In various embodiments, in an application in which the sample analyte of interest is a nucleic acid and downstream processing is amplification (e.g., PCR), a reagent additive that is another biological species or synthesized nucleic acid (e.g., DNA) with substantially the same ionic mobility of the sample nucleic acid can be used. By way of nonlimiting example, fish DNA may be used as the reagent additive when the sample analyte is human DNA, and the mixture subject to ITP using the fluidic devices and workflows in accordance with various embodiments described herein. For example, fish DNA at 1 nanogram/microliter (ng/uL) can be mixed with human DNA at 0.1 μL. By utilizing another biological species or synthesized DNA when the sample analyte is DNA, downstream amplification and detection assay (such as PCR for example) can occur on the mixture of ions (i.e., the additive DNA and the analyte DNA) that are banded and collected after ITP. Because the amplification assay can be made to react specifically with the analyte DNA and not the additive DNA, the additive DNA would not interfere with such downstream processing.

Aside from fish DNA, any genomic DNA, including plant DNA, not of human origin, such as for example outside the Hominidae Family may be utilized. The type of DNA chosen will be dependent on the type of nucleic acid analyte of interest, as those skilled in the art would appreciate. In various embodiments, the ratio of the additive reagent to the analyte can be in a range of 0.1 to 1. In applications involving protein analyte purification and enrichment with optical detection, the additive reagent of approximately equivalent ionic mobility can be, for example, fluorophore-labeled micro-spherical particulates having a size, shape, and charge selected so as to mimic similar ionic mobility of protein analyte of interest. For non-fluorescent based detection, short peptide encapsulated in micelles with appropriate ionic mobility can be used. Downstream analysis methods can include PCR for nucleic acid analytes and ELISA for protein analytes.

The following examples were performed and provide data indicating the unexpected results of the various embodiments described herein. The examples provided below should not be considered as limiting of the scope of the present disclosure and disclosed embodiments in any way, but rather are supplemental of the same.

### EXAMPLES

Prototype fluidic devices and a system breadboard was used to test four types of sample using the format of the fluidic device as represented in FIGS. 6-8. The four types of samples were prepared for iontophoresis (ITP) testing: (1) model sample using pure human gDNA without lysis buffer, (2) buccal swab samples lysed with Rapid HIT lysis buffer, (3) mocked human blood swab samples lysed with Rapid HIT lysis buffer, and (4) human gDNA sample at low concentration mixed with fish gDNA.

Three functionalities of the fluidic device and system were verified: (1) sample enrichment by ITP in which an optical detection mechanism was used to monitor the formation and migration of concentrated DNA band via fluorescent DNA intercalating dye; (2) sample purification by ITP in which enriched and purified sample was withdrawn from the ITP fluidic device and subject to an amplification assay using the GlobalFiler™ IQC PCR Amplification Kit commercialized by Thermo Fisher Scientific (hereinafter referred to as GlobalFiler™ IQC assay run and CE (capillary electrophoresis) analysis; and (3) mixed species samples, in which optical detection was used to monitor the formation and migration of concentrated human/fish DNA band via fluorescent DNA intercalating dye which is otherwise not optically detectable when human DNA is not mixed with fish DNA. A GlobalFiler™ IQC assay was used to verify the existence of fish DNA and that the fish DNA does not interfere with the electropherogram profile of pure human DNA.

The chip prototypes were built using either 3D printing, soft lithography, hot embossing or injection molding using materials such as

[symbol]

-PMMA), polydimethylsiloxane (PDMS), and cyclic olefin co-polymer (COC). The device was formed with the channel layer covered with a optically transparent plastic backing and thin layer of pressure sensitive adhesive as described above with reference to FIG. 22B.

As the leading electrolyte, a buffer was prepared with diluted 1 M Tris-HCI at pH 8 using de-ionized water to 0.1 M Tris-HCI and 1.5X DAPI without tuning the pH value. As the trailing electrolyte, a buffer was prepared by diluting 100 mM HEPES (4-(2-hydroxyethyl)-1-piperazineethanesulfonic acid )-NaCI to 30 mM HEPES-NaCl without tuning the pH value.

Model sample was prepared by using pure human gDNA from Jurkat cell line in 30 mM HEPES-NaCl and 1.5% polydimethylacrylate (PDMA). Buccal swab sample was collected by swabbing cheeks 20 times on both sides of the cheek. The swab was then placed in a 1.5 mL tube with 400 uL Rapid HID lysis buffer and lysed at 90 degrees Celsius for 10 minutes. Mocked blood sample was prepared in 2 steps. First, fresh human blood was collected and preserved with EDTA. Two uL of blood aliquot was pipetted on a Nylon swab to create a blood spot. The swab was then placed in a Falcon tube and dried and stored at a 4 degrees Celsius temperature until usage. Upon tests, the blood spotted swab was placed in a 1.5 mL tube with 400 uL Rapid HIT lysing buffer and placed for 10 minutes on a heat block heated to 90 degrees Celsius. After cell lysing of both buccal swab and mocked blood samples, the swab was discarded, and 300 uL of crude cell lysate was transferred to another 1.5 mL tube and mixed with 35 uL, 15.5% PDMA. The mixed human/fish DNA sample was prepared at 0.05 ng/uL and 0.5 ng/uL human DNA (Jurkat) and fish DNA (salmon sperm) respectively with 1.5% PDMA in low TE buffer.

To perform the various tests, the fluidic device (having the configuration as depicted in the embodiment of FIGS. 6-8) and utilizing the overall system setup with the fluidic device placed on a metal plate as a heat sink with a table fan blowing air directly on the fluidic device. LED light at 365 nm was placed on the side of the separation channel near the collection region, penetrated through the fluidic device material, and illuminated the separation channel from the side. A fiber optics pointed in a perpendicular direction to the illuminating light was connected to a spectrometer to collect live signals, such as fluorescence from the dye-labeled ionic components. Three separate syringes with silicone tubing attached on the needles were used to load the leading electrolyte, trailing electrolyte, and sample. First, any air in the syringes and tubings were pushed out. Then, the silicone tubing was respectively attached with the syringes and the free ends of the tubing connected to the three inlets of the fluidic device.

Next, the loading of the sample, leading electrolyte, and trailing electrolyte occurred as outlined in the workflow described above with respect to FIGS. 8A-8G. After loading inlet channels and the separation channel, sample was then injected to the separation channel at a desired volume in a range from 20 μL to 100 μL.

Next, the trailing electrolyte reservoir was unplugged (i.e., the port and through hole placed in fluidic communication) and the trailing electrolyte buffer was primed until to fill the trailing electrolyte buffer reservoir to a desired volume, with the leading and trailing buffer reservoirs filled to approximately the same level.

A high voltage was then applied to the electrodes with a ramp from 0 Volts to 500 Volts at 500 Volts/second. The 500 Volts were then maintained for 7 minutes. Upon the arrival of the concentrated DNA band at the collection juncture, as observed via the optical detection mechanism (using fiber optics and a spectrometer), the application of voltage to the electrodes was ceased and 10 μL at 3 μL/sec was withdrawn from the fluidic device through the collection channel and port via the collection syringe/tubing.

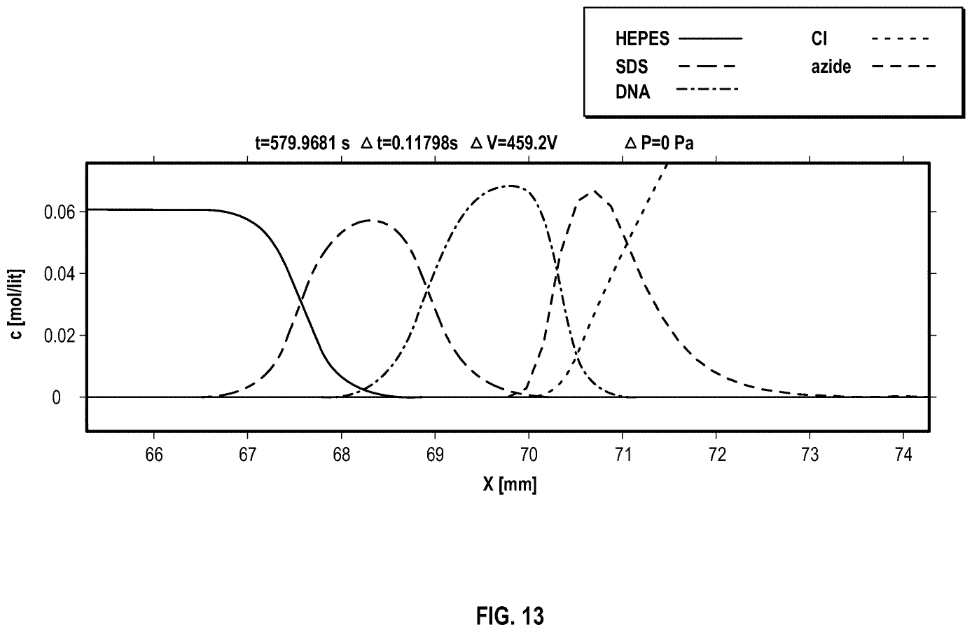

FIG. 13 shows computer generated simulation (using SPRESSO software) results of ITP using the sodium chloride with Tris buffer as the leading electrolyte and HEPES as the trailing electrolyte with genomic DNA mixed in lysis buffer composed of sodium lauryl sulfate (SDS) and sodium azide using a 3-phase loading configuration. Azide, DNA, and SDS remained in the sample phase while forming the separation of the 3 co-ionic species, that is chloride ion from the leading electrolyte, DNA, and HEPES from the trailing electrolyte. The graph of FIG. 13 plots the concentration of the various ionic components (e.g., chloride of the LE, HEPEs of the TE, and Azide, DNA, and SDS of the sample) in mols/liter (y-axis) along the length of separation (x-axis) in millimeters.

### Test Result 1

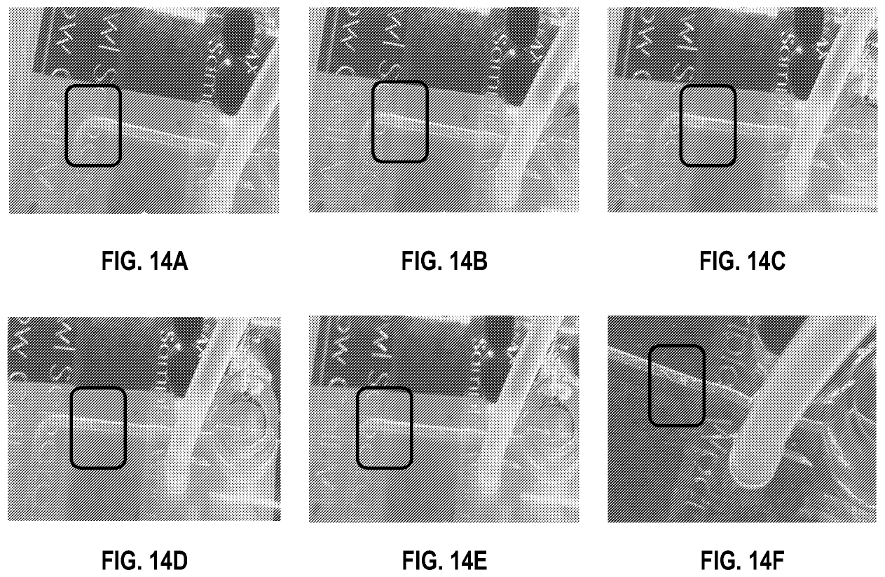

FIGS. 14A-14F show a series of time lapses of the same run of ITP sample using a fluidic device and sample, leading electrolyte, and trailing electrolyte as described in the Example above. The images are zoomed in to show the details of the separation channel near the intersection with collection channel (showing a portion of the device of FIG. 6 from where the separation channel bends and extends from the bottom of the U-shape to the collection channel and up to the leading electrolyte reservoir). When a volume of crude cell lysate sample is first introduced to the separation channel, it cannot be detected. As it travels toward the anode under application of applied voltage, a long segment with faint fluorescent signal starts to appear. The same plug becomes shorter and brighter and better defined as it travels further closer to anode. The segment/plug is circled in the photographs provided in FIGS. 14A-14F. The ITP was performed at 500V. It can be seen in this series of photographs how the analyte DNA molecules rearrange their relative location within the sample under ITP, transforming from a broad, relatively low concentration band to a narrower, relatively high concentration band.

### Test Result 2

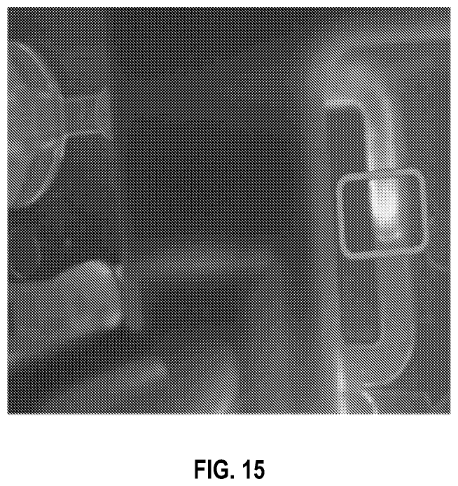

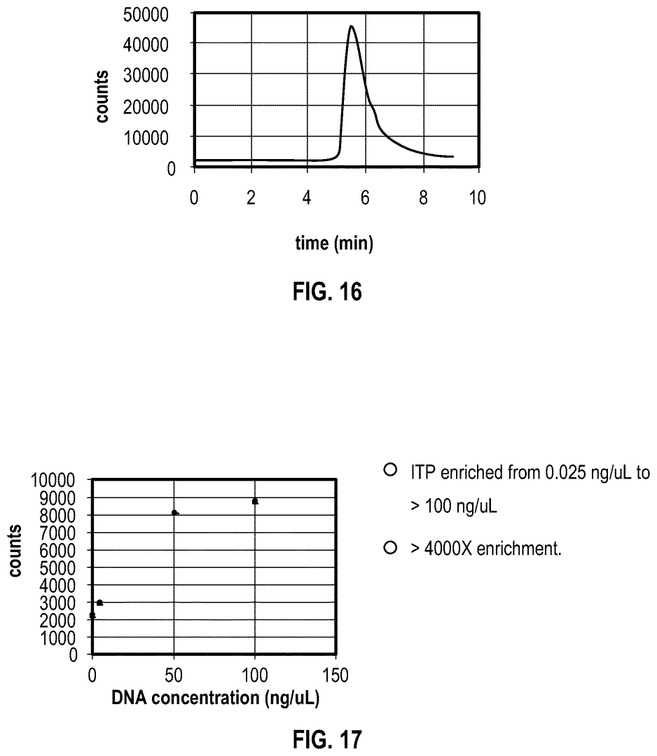

FIG. 15 shows how the DNA band formed without being skewed after it passes the first 90 degree bend of the U-shaped separation channel along the direction of the channel from the trailing electrolyte reservoir to the leading electrolyte reservoir. FIG. 15 also shows that the DNA band is free of air bubbles. As mentioned above, Joule heating was managed by blowing a table fan directly to the fluidic device. The graph in FIG. 16 depicts detected fluorescent signal strength (intensity measured in counts (photons) per second) at emmision wavelength 485 nm within one ITP run (fluorscence versus time in minutes being shown). The dye used in the test is DAPI (4′, 6-diamidino-2-phenylindole) using an exciation wavelength of 365 nm. As can be seen in FIG. 16, the signal began with low and steady background, then increased dramatically and declined back to initial state. The shape of the signal change suggests the passing of some fluorescent compound, in this case a DAPI-labeled DNA. FIG. 17 depicts a calibration curve of the fluorescent signal (counts versus concentration) corresponding to the separation channel having been filled with known concentration of DAPI-labeled DNA. The DNA concentration of the DNA band after enrichment was greater than 100 ng/uL, which implies the ITP forms some 4000 folds enrichment. The ITP for the results in FIGS. 15-17 were performed at the above-described protocol of 500V for 6 min.

### Test Result 3

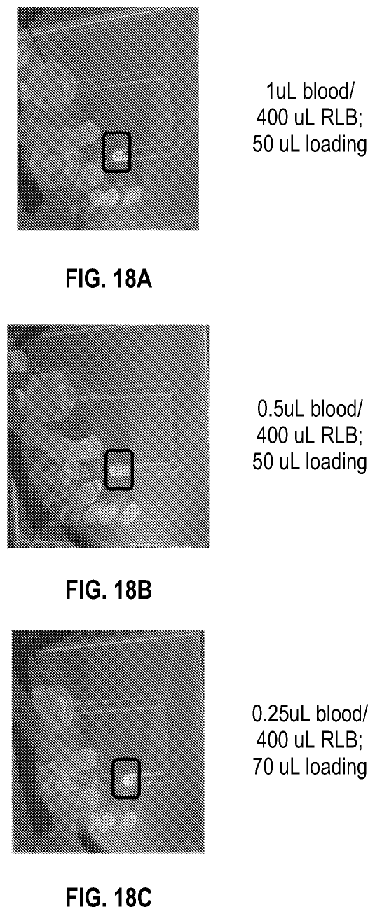

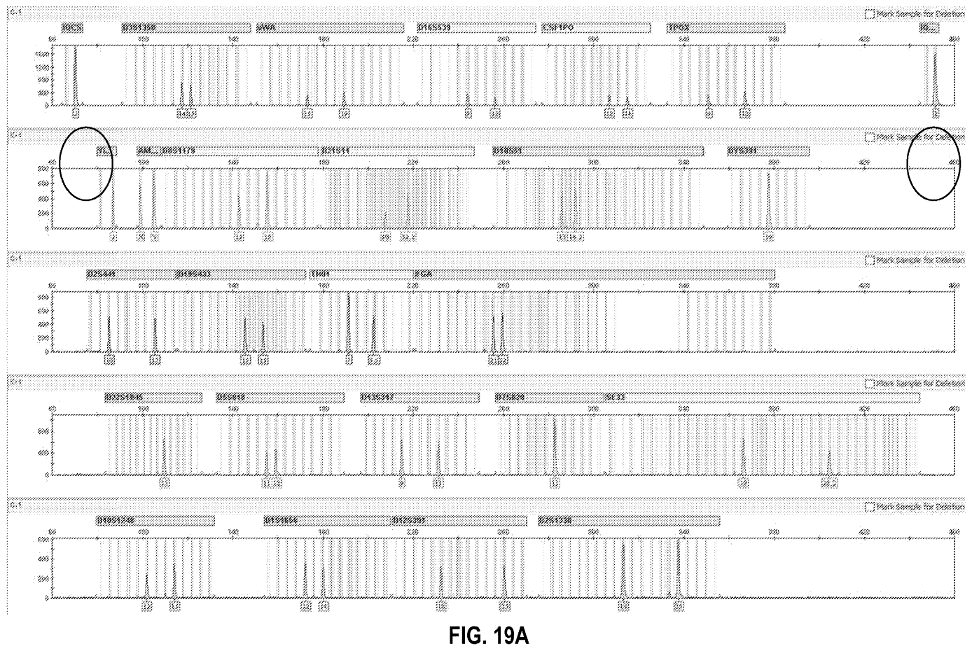

FIGS. 18A-18C depict images taken from tests using three differing concentrations of mocked blood sample dilution. The equivalent of 1 μL, 0.5 μL, and 0.25 μL blood on respective sample swabs were used and mixed with 400 μL Rapid lysis buffer (RLB). The lysed mixed sample was then loaded at 50 μL, 50 μL, and 70 μL respectively for the above blood samples. ITP was performed at 500V for 6 minutes, as described above. The images show the concentrated DNA band that occurs (the bright band in the channel where the arrows in the figures are pointing), thus demonstrating the device and method are capable of enriching biological samples previously lysed using surfactant.

### Test Result 4

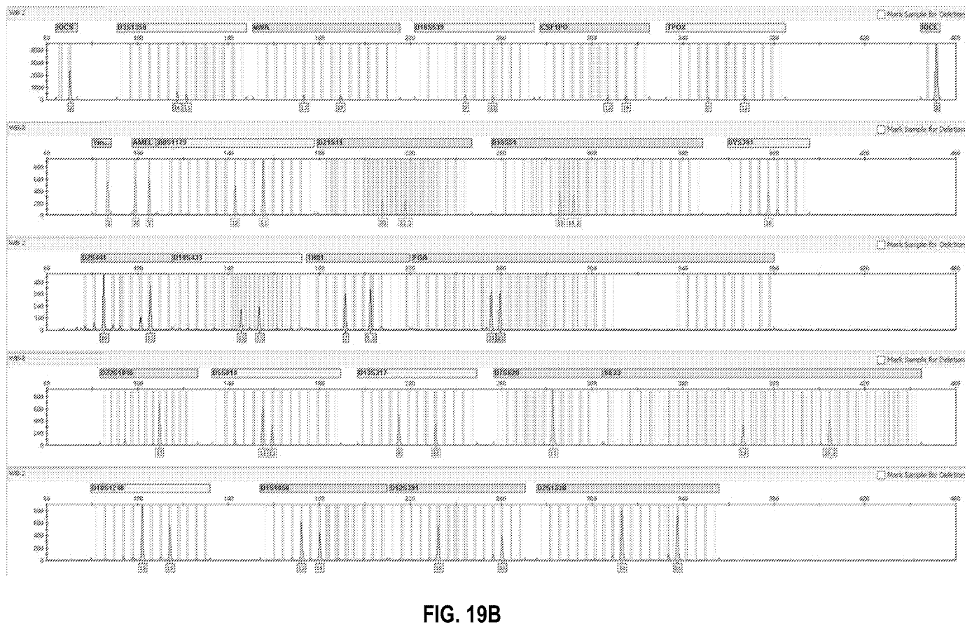

To test feasibility, a mocked blood sample was lysed using Rapid HIT lysis buffer. The lysate was then split to 2 aliquots for different processes. The first aliquot was used as the control sample and the second aliquot was used as ITP sample. The control sample was prepared by diluting the blood lysate 5 fold with trailing electrolyte and again 5 fold with MMX/PMX (Master Mix and Primer Mix) prior to PCR amplification. The ITP sample was prepared by loading 50 uL of the lysate to the fluidic device and performing ITP, collecting 10 uL of the enriched and purified sample from the fluidic device, and then mixing the collected encriched and purified sample with 15 uL of MMX/PMX prior to PCR amplification. Both control and ITP sample were then sent for down stream GlobalFiler IQC assay (an assay designed to amplify, fluorescently tag and analyze human identity by utilizing human DNA sample) and analyzed using capillary electrophoresis. Results are shown in FIGS. 19A and 19B, with FIG. 19A showing the electrophorogram of the control sample and FIG. 19B showing the electrophorogram of the collected enriched and purified ITP sample. As can be seen from the electrophorgrams, the height of 2 IQC control peaks in both figures present well, indicating the PCR inhibition is either insignificant or non-existing. All allelic peaks inditified in the control panel also are present in the ITP sample panel. The results demonstrate ITP using the fluidic device successfully enriched and purified the sample with PCR inhibitor. Short tandem repeats (STR) also are identified in the figures.

### Test Result 5

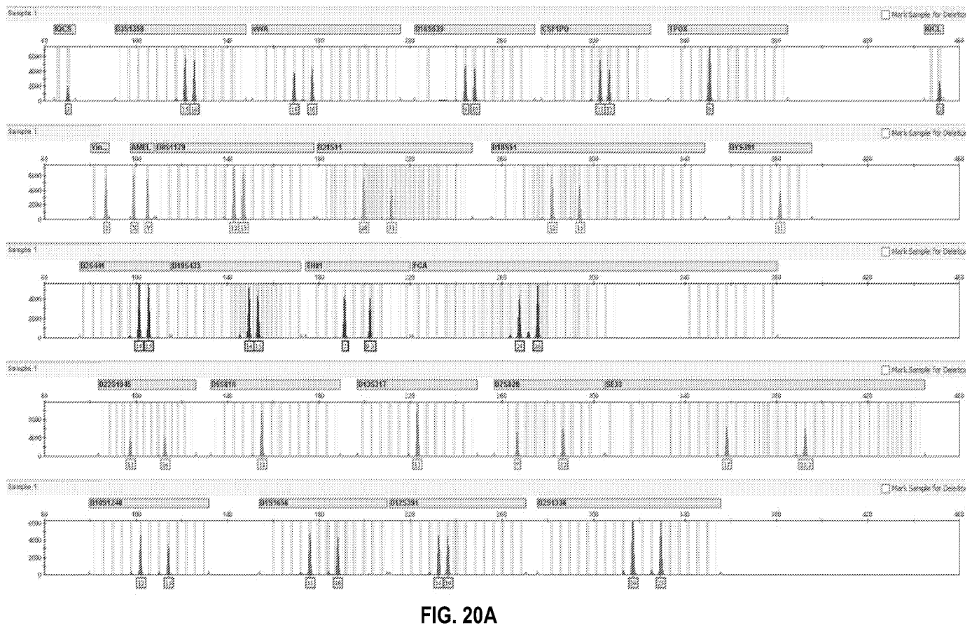

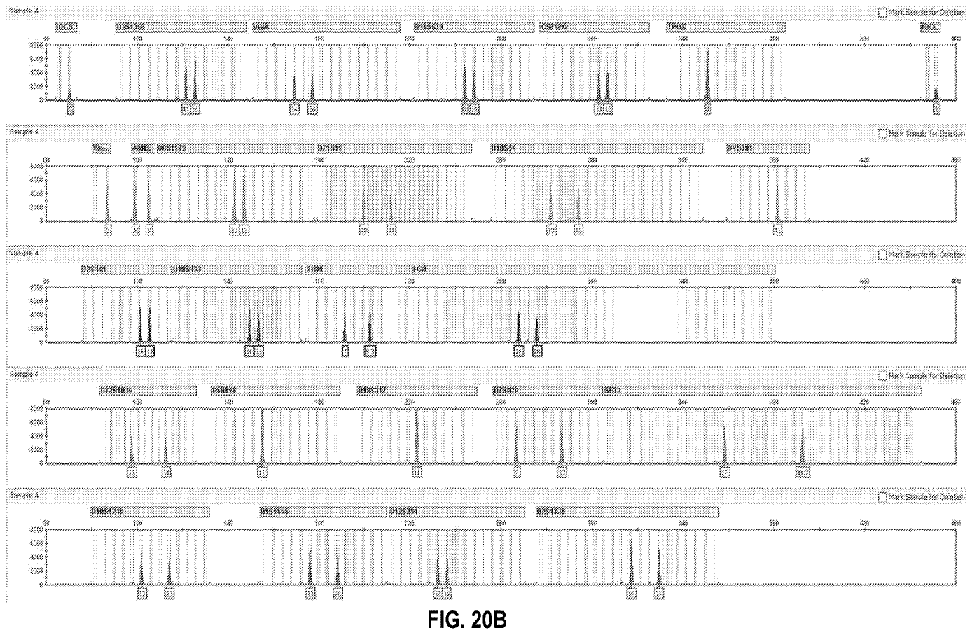

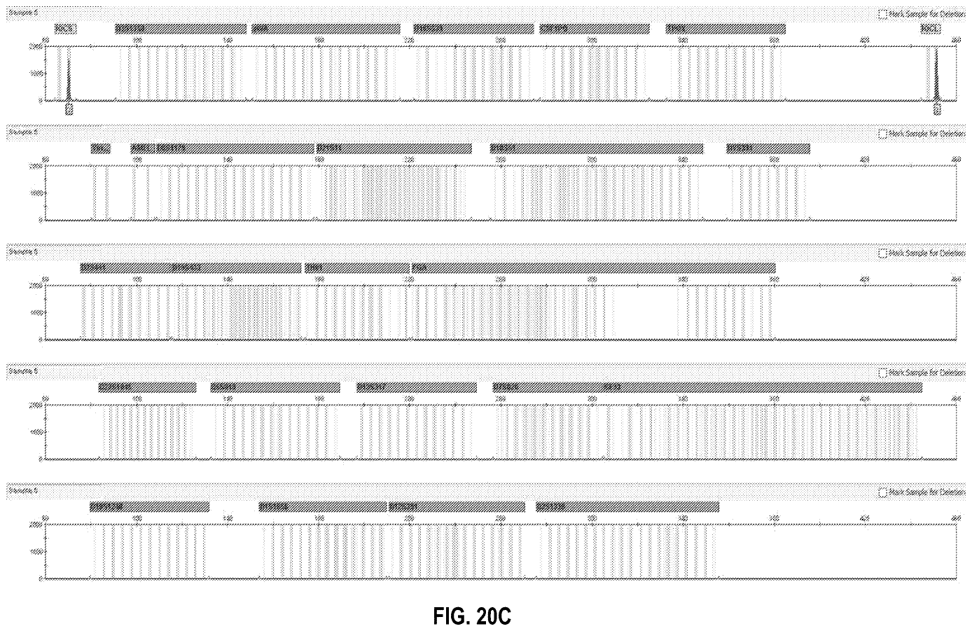

To further test feasibility tests using pure human DNA, mixed human/salmon DNA, and pure salmon DNA were conducted with GlobalFiler™ IQC assay, using fish/human DNA in a ratio of 1:10. Results are shown in FIGS. 20A-20C and demonstrate that the GlobalFiler™ IQC assay can faithfully express human sample profile even when mixed with salmon DNA. No difference on STR profile is observed between pure human DNA sample (FIG. 20A) and human/salmon DNA sample (FIG. 20B). In addition, FIG. 20C shows that the fish DNA was not expressed using GlobalFiler™ IQC assay.

### Test Result 7

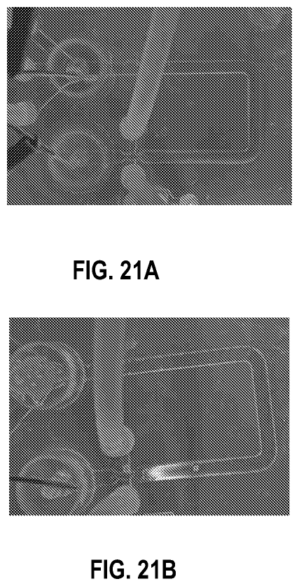

An additional feasibility test was performed using pure human DNA and mixed human/salmon DNA using ITP in the fluidic device as described in the Example. 1 uL blood swab lysed in 400 uL Rapid lysis buffer/ 1.5% PDMA was prepared and the lysate diluted 20 fold to make an equivalent concentration of 0.05 ul blood. Mixed human/salmon sample was prepared by diluting the same blood lysate 40X in Rapid lysis buffer/1.5% PDMA/salmon sperm DNA at 0.05 ng/uL concentration. Both the control and mixed samples were separately loaded at 50 uL into a fluidic device and ITP run at 500 V for 6 minutes. FIG. 21A shows the control sample test image. As shown the band formation is not visible in FIG. 21A. FIG. 21B shows the mixed human/salmon sample and the visible resulting DNA band formation (see arrow) of purified and enriched mixed sample.

This description and the accompanying drawings that illustrate various embodiments should not be taken as limiting. Various mechanical, compositional, structural, electrical, and operational changes may be made without departing from the scope of this description and claims, including equivalents. In some instances, well-known structures and techniques have not been shown or described in detail so as not to obscure the disclosure. For example, the various inlet channels of the fluidic devices in certain embodiments can be used to alternatively load differing ones of the sample, leading electrolyte, and trailing electrolyte as would be understood by those having ordinary skill in the art. In various figures, like numbers in two or more figures with a different series may represent the same or similar elements, and an effort has been made to state the same when such is the case. Furthermore, elements and their associated features that are described in detail with reference to one embodiment may, whenever practical, be included in other embodiments in which they are not specifically shown or described. For example, if an element is described in detail with reference to one embodiment and is not described with reference to another embodiment, the element may nevertheless be claimed as included in the other embodiment.

For the purposes of this specification and appended claims, unless otherwise indicated, all numbers expressing quantities, percentages, or proportions, and other numerical values used in the specification and claims, are to be understood as being modified in all instances by the term “about” or “approximately” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to be limiting of the scope of the disclosure and claims. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. In the context of flow, proximal may refer to portions of a device through which fluid is flowing that are upstream of other portions; likewise distal may refer to portions of the device through which fluid is flowing that are downstream from other portions.

Further modifications and alternative embodiments will be apparent to those of ordinary skill in the art in view of the disclosure herein. For example, the devices, systems, and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

For example, it is contemplated as within the scope of the present disclosure that various systems, devices, and techniques can be used herein to perform multiplexed ITP assays in which multiple target ionic analytes of interest (e.g., multiple different nucleic acids and/or proteins of a sample) are to be purified and enriched. Moreover, while various of the workflows were described as performing isotachophoresis to migrate negatively charged ionic components, including ionic analytes, along a separation channel, those having ordinary skill in the art would understand from the principles and description herein how to modify the various fluidic devices, leading and trailing electrolytes, applied electric field, and workflows associated therewith to perform isotachophoresis to purify and enrich negatively charged ionic components, including ionic analytes.

In the context of the present invention, at least the following embodiments are described.

Embodiment 1 is an apparatus comprising a substrate comprising a first portion and a second portion separated from each other by a cross-sectional plane that is perpendicular to a thickness dimension of the substrate. The first portion comprises a plurality of inlet channels each comprising an inlet and an outlet, the outlets of the plurality of inlet channels meeting at a juncture, and an intermediate channel extending from the juncture to a location spaced from the juncture. The second portion comprises a first reservoir fluidically coupled to the plurality of inlet channels and the intermediate channel at the juncture, and a second reservoir fluidically coupled to the intermediate channel at the location spaced from the juncture. The apparatus further comprises a first electrode configured to be in electrically conductive communication with the first reservoir and a second electrode configured to be in electrically conductive communication with the second reservoir.

Embodiment 2 is an apparatus that comprises a substrate defining a fluidic network, the fluidic network comprising a plurality of inlet channels each comprising an inlet and an outlet, the outlets of the inlet channels meeting at a juncture, and the inlets of the inlet channels being separated from each other and configured to be placed in flow communication with one or more components to receive substances to introduce to the fluidic network, an intermediate channel extending from the juncture and fluidically coupled to outlets of the plurality of inlet channels, a first reservoir fluidically coupled to the plurality of inlet channels and the intermediate channel at the juncture, and a second reservoir fluidically coupled to the intermediate channel at a location downstream of the juncture. The inlet channels and the intermediate channel are co-planar, and the first reservoir and second reservoir are configured to produce substantially equal pressure heads.

The apparatus comprises a first electrode in electrically conductive communication with the first reservoir; and a second electrode in electrically conductive communication with the second reservoir.

Embodiment 3 is the apparatus of any of embodiment 1 or 2, wherein the apparatus further comprises a collection channel intersecting and fluidically coupled to the intermediate channel proximate the second reservoir, the collection channel configured to be fluidically coupled to one or more components to remove substances from the fluidic network, and the collection channel being coplanar with the inlet and intermediate channels.

Embodiment 4 is the apparatus of any of embodiment 1 or 2, wherein the apparatus further comprises a first port fluidically with the first reservoir and with the plurality of inlet channels and the intermediate channel.

Embodiment 5 is the apparatus of embodiment 4, wherein the first port is fluidically coupled to the plurality of inlet channels and the intermediate channel via a through hole.

Embodiment 6 is the apparatus 5, further comprising a valve configured to selectively open or close flow communication from the through hole through the first port.

Embodiment 7 is the apparatus of embodiment 4, further comprising a second port fluidically coupled with the second reservoir and with the intermediate channel at a second juncture.

Embodiment 8 is the apparatus of either claim 1 or 2, wherein the substrate has a length to width ratio ranging from 0.7 to 1.4.

Embodiment 9 is the apparatus of embodiment 2, wherein a ratio of a length of the intermediate channel from the first reservoir to the collection channel to a length of the intermediate channel from the first reservoir to the second reservoir ranges from 0.7 to 1.0.

Embodiment 10 is the apparatus of embodiment 1 or 2, wherein first and second reservoirs are at a same location along length dimension of substrate and spaced from each other along a width dimension of substrate.

Embodiment 11 is the apparatus of embodiment 10, wherein a center-to-center distance between the first and second reservoirs along the width dimension of the substrate ranges from 5 mm to 50 mm.

Embodiment 12 is the apparatus of emobidment 1 or 2, wherein the first and second reservoirs are each configured to contain liquid so as to provide a substantially equal pressure head to the intermediate channel.

Embodiment 13 is the apparatus of embodiment 1, further comprising an optical detection mechanism configured to detect electromagnetic emission from the intermediate channel.

Embodiment 14 is a method of processing a biological sample. The method comprises forming a biological sample mixture by mixing a biological sample containing an ionic analyte with an additive reagent containing an ionic component different than the ionic analyte, and subjecting the mixture to isotachophoresis.

The the ionic analyte and the ionic component exhibit substantially equivalent ionic mobility and the ionic component does not interfere with an analysis assay performed on the ionic analyte after isotachophoresis.

Embodiment 15 is the method of embodiment 14, wherein the ionic mobility of the ionic analyte and the ionic mobility of the ionic component are within a range of +/−5% of each other.

Embodiment 16 is a method of processing a biological sample. The method comprises introducing into a channel a biological sample containing an ionic analyte and additive reagent containing an ionic component different than the ionic analyte; and using an electric field to cause migration of the ionic analyte and ionic component along the channel. The migration of the ionic analyte and ionic component occurs at approximately a same rate and results in separation of the ionic analyte and ionic component from one or more other components of the biological sample.

Embodiment 17 is the method of embodiment 14 or 16, wherein the biological sample is from a first biological species and the additive reagent is from a second biological species.

Embodiment 18 is the method of embodiment 16, wherein the first biological species is human and the second biological species is fish.

Embodiment 19 is the method of embodiment 14 or 16, wherein the ionic analyte is nucleic acid and the ionic component of the additive reagent is synthesized nucleic acid.

Embodiment 20 is the method of embodiment 14 or 16, wherein subjecting the mixture to isotachophoresis causes the ionic analyte and the ionic component to separate from one or more other components having differing ionic mobility than an ionic mobility of the ionic analyte and the ionic component.

Embodiment 21 is the method of embodiment 14 or 16, wherein the ionic component does not interfere with an amplification assay designed to amplify the ionic analyte.

Embodiment 22 is the method of embodiment 21, wherein the amplification assay comprises one or more of polymerase chain reaction (PCR), sequencing, or enzyme-linked immunoassay (ELISA).

Embodiment 23 is the method of embodiment 16, wherein the electric field is created by applying a 500V electric potential to electrodes.

Embodiment 24 is the method of embodiment 14 or 16, further comprising detecting electromagnetic radiation emission from the ionic analyte.

Embodiment 25 is a method for performing isotachophoresis. The method comprises loading a separation channel with a first electrolyte and a biological sample containing an ionic analyte in series along a length of the separation channel, wherein the loading to the separation channel is through differing channels, an inlet of the separation channel and an outlet of at least a first inlet channel through which the sample is introduced are fluidically coupled to a first reservoir via a first through hole, with the first reservoir containing a second electrolyte and with a second reservoir fluidically coupled at an outlet of the separation channel containing the first electrolyte, applying an electric field between the first and second reservoirs and thereby causing: migration of ionic components including the ionic analyte of the biological sample along the separation channel in a direction from first reservoir to the second reservoir, and separation of the ionic analyte from one or more other ionic components of the sample. The first electrolyte exhibits a first ionic mobility, the second electrolyte exhibits a second ionic mobility, and the ionic analyte exhibits a third ionic mobility, the first ionic mobility being higher than the third ionic mobility, and the third ionic mobility being higher than the second ionic mobility.

Embodiment 26 is the method of embodiment 25, further comprising collecting the ionic analyte separated from the one or more other ionic components from the separation channel.

Embodiment 27 is the method of embodiment 25, further comprising detecting the ionic analyte separated from the one or more other ionic components in the separation channel.

Embodiment 28 is the method of embodiment 25, further comprising introducing with the biological sample and an additive reagent having an ionic mobility substantially the same as an ionic mobility of the ionic analyte.

Embodiment 29 is the method of embodiment 25, further comprising loading the second electrolyte through a second inlet channel fluidically coupled to the first reservoir via the first through hole, the separation channel, and the first inlet channel.

Embodiment 30 is the method of embodiment 29, further comprising loading the first electrolyte through a third inlet channel fluidically coupled to the first reservoir via the first through hole, the separation channel, and the first and second inlet channels.

Embodiment 31 is the method of embodiment 30, further comprising, prior to loading the sample into the separation channel: priming the first inlet channel with the sample, priming the second inlet channel with the second electrolyte, closing a valve to close flow communication between the first reservoir via the first through hole with each of the first inlet channel, the second inlet channel, the third inlet channel and the separation channel, and priming the third inlet channel, the second reservoir, and the separation channel with the first electrolyte.

Embodiment 32 is the method of embodiment 29, further comprising loading the first electrolyte through a third inlet channel fluidically coupled to the second reservoir via a second through hole and the outlet of the separation channel.

Embodiment 33 is the method of embodiment 32, further comprising prior to loading the sample into the separation channel: priming the first inlet channel with sample, priming the second inlet channel with the second electrolyte, priming the third inlet channel, the second reservoir, and the separation channel with the first electrolyte, and closing a valve to close flow communication between the first reservoir via the first through hole and each of the first inlet channel, the second inlet channel, and the separation channel.

Embodiment 34 is the method of embodiment 31 or 33, further comprising, after loading sample into the separation channel, opening the valve and priming the first reservoir with the second electrolyte.

Embodiment 35 is the method of embodiment 25, wherein a level of the second electrolyte in the first reservoir and a level of the first electrolyte in the second reservoir are within a range of about 3 mm of each other.

Embodiment 36 is the method of embodiment 25, wherein the first reservoir containing the second electrolyte and the second reservoir containing the first electrolyte supply a substantially equal pressure head to the separation channel.

Embodiment 37 is a method comprising priming a first inlet channel with a volume of biological sample comprising an ionic analyte to fill the first inlet channel; priming a second inlet channel with a volume of first electrolyte to fill the second inlet channel; priming a separation channel with a volume of second electrolyte to fill the separation channel, the separation channel being fluidically coupled to the first inlet channel and the second inlet channel at a juncture; with a through hole at the juncture configured to vent the first inlet, second inlet, and separation channels in a closed state, loading the biological sample from the first inlet channel into the separation channel; and with the through hole in an open state: loading the first electrolyte from the second inlet channel into a first reservoir fluidically coupled to the separation channel via the through hole, and applying an electrical field to cause migration of ionic components through the separation channel. The first electrolyte exhibits a first ionic mobility, the second electrolyte exhibits a second ionic mobility, and the ionic analyte exhibits a third ionic mobility, the second ionic mobility being higher than the third ionic mobility, and the third ionic mobility being higher than the first ionic mobility.

Embodiment 38 is the method comprising forming a biological sample mixture by mixing a biological sample containing an ionic analyte with an additive reagent configured to alter a viscosity of the biological sample; loading a channel with an electrolyte and a biological sample mixture; and applying an electrical field to the channel and thereby causing: migration of ionic components including the ionic analyte of the biological sample along the channel, and separation of the ionic analyte from one or more ionic components of the electrolyte. The electrolyte has a first ionic mobility, and the ionic analyte has a second ionic mobility, the first ionic mobility being higher than the second ionic mobility. The electrolyte has a first viscosity and the biological sample mixture has a second viscosity, and the first and second viscosities differing from each other.

Embodiment 39 is the method of embodiment 38, wherein the second viscosity ranges from 2 centipoise to 10 centipoise.

Embodiment 40 is the method of embodiment 38, wherein the first and second viscosities differ from each other by 1 centipoise to 10 centipoise.

Embodiment 41 is the method of embodiment 38, wherein the biological sample mixture comprises a biological sample containing the ionic analyte and an additive reagent having a viscosity that is greater than the biological sample.

Embodiment 42 is the method of embodiment 41, wherein the additive reagent comprises polydimethylacrylate (PDMA), Ficoll, Dextran, or any combination thereof.

It is to be understood that the particular examples and embodiments set forth herein are non-limiting, and modifications to structure, dimensions, materials, and methodologies may be made without departing from the scope of the present teachings.

The entire contents of all cited references in this disclosure are incorporated herein for all purposes. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure or claims be limited to the specific values recited when defining a range.

Although the disclosure has been described in detail with particular reference to the described embodiments and examples, other embodiments can achieve the same or similar results. Variations and modifications of those embodiments provided will be evident to one of ordinary skill in the art and the disclosure covers all such modifications and equivalents. The specification and examples are to be considered exemplary only and are not limiting to the claims.

1. An apparatus comprising:
a substrate comprising a first portion and a second portion separated from each other by a cross-sectional plane that is perpendicular to a thickness dimension of the substrate,
wherein the first portion comprises:
a plurality of inlet channels each comprising an inlet and an outlet, the outlets of the plurality of inlet channels meeting at a juncture,
an intermediate channel extending from the juncture to a location spaced from the juncture,
wherein the second portion comprises:
a first reservoir fluidically coupled to the plurality of inlet channels and the intermediate channel at the juncture, and
a second reservoir fluidically coupled to the intermediate channel at the location spaced from the juncture;
a first electrode configured to be in electrically conductive communication with the first reservoir; and
a second electrode configured to be in electrically conductive communication with the second reservoir.

2. An apparatus comprising:
a substrate defining a fluidic network, the fluidic network comprising:
a plurality of inlet channels each comprising an inlet and an outlet, the outlets of the inlet channels meeting at a juncture, and the inlets of the inlet channels being separated from each other and configured to be placed in flow communication with one or more components to receive substances to introduce to the fluidic network,
an intermediate channel extending from the juncture and fluidically coupled to outlets of the plurality of inlet channels,
a first reservoir at the juncture and fluidically coupled to the inlet channels and the intermediate channel,
a second reservoir fluidically coupled to the intermediate channel at a location downstream of the juncture,
wherein the inlet channels and the intermediate channel are co-planar, and wherein the first reservoir and second reservoir are configured to produce substantially equal pressure heads; and
a first electrode in electrically conductive communication with the first reservoir; and
a second electrode in electrically conductive communication with the second reservoir.

3. The apparatus of claim 1, further comprising a collection channel intersecting and fluidically coupled to the intermediate channel proximate the second reservoir, the collection channel configured to be fluidically coupled to one or more components to remove substances from the fluidic network, and the collection channel being coplanar with the inlet and intermediate channels.

4. The apparatus of claim 1, further comprising a first port fluidically with the first reservoir and with the plurality of inlet channels and the intermediate channel.

5. The apparatus of claim 4, wherein the first port is fluidically coupled to the plurality of inlet channels and the intermediate channel via a through hole.

6. The apparatus of claim 5, further comprising a valve configured to selectively open or close flow communication from the through hole through the first port.

7. The apparatus of claim 4, further comprising a second port fluidically coupled with the second reservoir and with the intermediate channel at a second juncture.

8. The apparatus of claim 1, wherein the substrate has a length to width ratio ranging from 0.7 to 1.4.

9. The apparatus of claim 2, wherein a ratio of a length of the intermediate channel from the first reservoir to the collection channel to a length of the intermediate channel from the first reservoir to the second reservoir ranges from 0.7 to 1.0.

10. The apparatus of claim 1, wherein first and second reservoirs are at a same location along length dimension of substrate and spaced from each other along a width dimension of substrate.

11. The apparatus of claim 10, wherein a center-to-center distance between the first and second reservoirs along the width dimension of the substrate ranges from 5 mm to 50 mm.

12. The apparatus of claim 1 wherein the first and second reservoirs are each configured to contain liquid so as to provide a substantially equal pressure head to the intermediate channel.

13. The apparatus of claim 1, further comprising an optical detection mechanism configured to detect electromagnetic emission from the intermediate channel.

14-15. (canceled)

16. A method of processing a biological sample, the method comprising:
introducing into a channel a biological sample containing an ionic analyte and additive reagent containing an ionic component different than the ionic analyte; and
using an electric field to cause migration of the ionic analyte and ionic component along the channel,
wherein the migration of the ionic analyte and ionic component occurs at approximately a same rate and results in separation of the ionic analyte and ionic component from one or more other components of the biological sample.

17. The method of claim 16, wherein the biological sample is from a first biological species and the additive reagent is from a second biological species.

18. The method of claim 16, wherein the first biological species is human and the second biological species is fish.

19. The method of claim 16, wherein the ionic analyte is nucleic acid and the ionic component of the additive reagent is synthesized nucleic acid.

20. The method of claim 16, wherein subjecting the mixture to isotachophoresis causes the ionic analyte and the ionic component to separate from one or more other components having differing ionic mobility than an ionic mobility of the ionic analyte and the ionic component.

21. The method of claim 16, wherein the ionic component does not interfere with an amplification assay designed to amplify the ionic analyte.

22. The method of claim 21, wherein the amplification assay comprises one or more of polymerase chain reaction (PCR), sequencing, or enzyme-linked immunoassay (ELISA).

23-42. (canceled)
